(19) United States
(12) Patent Application Publication
(10) Pub. No.: US 2026/0341486 A1
(43) Pub. Date: Oct. 29, 2026

(54) DIALOGUE-AWARE RESPIRATORY EVENT DETECTION IN A CONVERSATIONAL ARTIFICIAL INTELLIGENCE (AI) SYSTEM

(71) Applicant: HealthGPT, Inc. DBA Hippocratic AI, Palo Alto, CA (US)

(72) Inventors: Vishal PARIKH, Palo Alto, CA (US); Kriti Aggarwal, Redmond, WA (US); Munjal SHAH, Palo Alto, CA (US)

(73) Assignee: HealthGPT, Inc. DBA Hippocratic AI, Palo Alto, CA (US)

(21) Appl. No.: 19/715,256

(22) Filed: Jun. 22, 2026

Related U.S. Application Data
(63) Continuation-in-part of application No. 18/900,289, filed on Sep. 27, 2024.
Application No. 18/900,289 is a continuation of application No. 18/592,441, filed on Feb. 29, 2024, now Pat. No. 12,142,371.
(60) Provisional application No. 63/466,712, filed on May 15, 2023.
(60) Provisional application No. 63/611,762, filed on Dec. 18, 2023.

(51) Int. Cl. G16H 40/20; G10L 15/18; G10L 15/183; G10L 15/19; G10L 15/22; G10L 25/66

(52) U.S. Cl. CPC G16H 40/20; G10L 15/183; G10L 15/22; G10L 25/66; G10L 15/1822; G10L 15/19

(57) ABSTRACT

In some aspects, a system receives an audio stream of a conversation between an artificial intelligence (AI) and a patient. The audio stream includes speech and may include respiratory artifacts. An AI agent processes the speech while conducting the conversation with the patient. While conducting the conversation, the AI agent (or a second AI agent), in parallel, may analyze the respiratory artifacts to detect respiratory events and predict an event indicator, an event type classification, an event duration, a confidence score, and a severity score. A gating mechanism may apply one or more gating rules to the event prediction to determine an output action. Based on the output action, the AI agent adapts its conversational behavior, for example by transmitting a directed elicitation instruction to the patient to obtain additional audio for analysis, or by transferring the conversation to a medical professional.

### PRIORITY DATA

The present non-provisional patent application claims priority to and the benefit from U.S. Application Ser. No. 18/900,289 filed on Sep. 27, 2024, which is incorporated herein by reference in its entirety and for all purposes as if completely and fully set forth herein.

### BACKGROUND OF THE TECHNOLOGY DISCLOSED

### Field of the Technology Disclosed

The technology disclosed relates to artificial intelligence type computers and digital data processing systems and corresponding data processing methods and products for emulation of intelligence (e.g., knowledge based systems, reasoning systems, and knowledge acquisition systems); and including systems for reasoning with uncertainty (e.g., fuzzy logic systems), adaptive systems, machine learning systems, and artificial neural networks. In particular, the technology disclosed relates generally to systems and techniques to provide a low-latency conversational artificial intelligence (AI) architecture with a parallelized in-depth analysis feedback loop for healthcare-related applications.

### Description of the Related Art

Current respiratory sound detection systems typically operate as standalone machine learning classifiers that analyze recorded audio to identify events such as coughs or breathing anomalies. These systems typically use specialized equipment and are primarily designed for offline analysis or passive monitoring. Meanwhile, emerging conversational AI systems, such as voice assistants and large language model-based dialogue agents, process speech primarily for linguistic content and while ignoring physiological acoustic signals embedded within the audio stream. As a result, existing conversational agents lack the ability to interpret respiratory events occurring during user interactions or to incorporate such signals into conversational reasoning and response generation.

### SUMMARY OF THE TECHNOLOGY DISCLOSED

This Summary provides a simplified form of concepts that are further described below in the Detailed Description. This Summary is not intended to identify key or essential features and should therefore not be used for determining or limiting the scope of the claimed subject matter.

In some aspects, a system receives an audio stream of a conversation between an artificial intelligence (AI) and a patient. The audio stream includes speech and may include respiratory artifacts. An AI agent processes the speech while conducting the conversation with the patient. While conducting the conversation, the AI agent (or a second AI agent), in parallel, may analyze the respiratory artifacts to detect respiratory events and predict an event indicator, an event type classification, an event duration, a confidence score, and a severity score. A gating mechanism may apply one or more gating rules to the event prediction to determine an output action. Based on the output action, the AI agent adapts its conversational behavior, for example by transmitting a directed elicitation instruction to the patient to obtain additional audio for analysis, or by transferring the conversation to a medical professional

### BRIEF DESCRIPTION OF THE DRAWINGS

A more complete understanding of the present disclosure may be obtained by reference to the following Detailed Description when taken in conjunction with the accompanying Drawings. In the figures, the left-most digit(s) of a reference number identifies the figure in which the reference number first appears. The same reference numbers in different figures indicate similar or identical items.

FIG. 1 is a block diagram of a system illustrating large language models (LLMs) to perform specialized healthcare-related functions, according to some implementations.

FIG. 2 is a block diagram illustrating an artificial intelligence (AI) architecture, according to some implementations.

FIG. 3 is a block diagram of a system that includes a low-latency conversational artificial intelligence (AI) architecture with a parallelized in-depth analysis and feedback loop, according to some implementations.

FIG. 4 is a block diagram of a system in which, during a conversation between a human and an artificial intelligence (AI) agent, respiratory artifacts in an audio stream of the conversation are detected, according to some implementations.

FIG. 5 is a block diagram illustrating exemplary respiratory events that are detected, according to some implementations.

FIG. 6 is a block diagram illustrating requests that may be made to a human after a respiratory artifact is detected, according to some implementations.

FIG. 7 is a flowchart of a process that includes analyzing a human response using a second opinion module, according to some implementations.

FIG. 8 is a flowchart of a process that includes accessing a conversation summary, a knowledge graph, or both, according to some implementations.

FIG. 9 is a flowchart of a process to train a machine learning algorithm, according to some implementations.

FIG. 10 illustrates an example configuration of a computing device that can be used to implement the systems and techniques described herein.

FIG. 11 is a flowchart of a process that applies a gating mechanism to an event prediction to determine an output action, according to some implementations.

FIG. 12 is a flowchart of a process that includes an artifact artificial intelligence (AI) agent instructing a conversational AI agent, according to some implementations.

FIG. 13 is a flowchart of a process that includes determining whether to escalate a conversation to a medical professional, according to some implementations.

### DETAILED DESCRIPTION

The technology disclosed can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

One or more implementations and clauses of the technology disclosed, or elements thereof can be implemented in the form of a computer product, including a non-transitory computer readable storage medium with computer usable program code for performing the method steps indicated. Furthermore, one or more implementations and clauses of the technology disclosed, or elements thereof can be implemented in the form of an apparatus including a memory and at least one processor that is coupled to the memory and operative to perform exemplary method steps. Yet further, in another aspect, one or more implementations and clauses of the technology disclosed or elements thereof can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

The clauses described in this section can be combined as features. In the interest of conciseness, the combinations of features are not individually enumerated and are not repeated with each base set of features. The reader will understand how features identified in the clauses described in this section can readily be combined with sets of base features identified as implementations in other sections of this application. These clauses are not meant to be mutually exclusive, exhaustive, or restrictive; and the technology disclosed is not limited to these clauses but rather encompasses all possible combinations, modifications, and variations within the scope of the claimed technology and its equivalents.

Other implementations of the clauses described in this section can include a non-transitory computer readable storage medium storing instructions executable by a processor to perform any of the clauses described in this section. Yet another implementation of the clauses described in this section can include a system including memory and one or more processors operable to execute instructions, stored in the memory, to perform any of the clauses described in this section.

As a first example, a computer-implemented method (“method”) is performed by a computer having one or more processors. The method includes receiving an audio stream from a patient and separating the audio stream into a speech stream and an artifact stream. A first artificial intelligence (AI) agent processes the speech stream to engage in a conversation with the patient. A second AI agent, operating in parallel with the first AI agent, performs an analysis of the artifact stream, to detect a respiratory acoustic event. The second AI agent determines an event prediction comprising an event indicator, an event type classification, an event duration, a confidence score, or a severity score for the respiratory acoustic event. A gating mechanism may apply one or more gating rules to the event prediction to determine an output action. Based on determining that the output action satisfies an escalation rule, the conversation is escalated by transferring the conversation to a medical professional, automatically initiating a call to emergency services, or both. Applying the one or more gating rules may include applying a temporal suppression rule that prevents the gating mechanism from triggering the output action within a suppression window following a prior trigger and applying a cooldown interval rule that enforces a minimum time interval between consecutive directed elicitation actions of a same type. An elicitation instruction may be sent from the second AI agent to the first AI agent, causing the first AI agent to perform a directed elicitation action by incorporating the elicitation instruction into the conversation. The second AI agent may analyze the audio produced by the patient in response to the directed elicitation action to produce an updated event prediction. For example, the directed elicitation action may include at least one of: requesting the patient to produce a voluntary cough, requesting the patient to speak a particular word or phrase selected to isolate respiratory acoustic artifacts, requesting the patient to assume a particular physical position to redistribute fluid in airways, requesting the patient to reposition a microphone-bearing device to improve audio capture quality, requesting the patient to move to a new location having less ambient noise than a current location, requesting the patient to switch to a higher-bandwidth communication medium to improve an audio quality of the artifact stream, or any combination thereof. The method may include receiving, by the second AI agent, contextual conversational information from the first AI agent. For example, the contextual conversational information may include at least one of a conversation transcript, a conversation summary, or an electronic health record data of the patient. The second AI agent may include the contextual conversational information in the analysis of the artifact stream to detect the respiratory acoustic event. The method may include receiving biometric data from a wearable device associated with the patient and correlating, by the second AI agent, the biometric data with the respiratory acoustic event to produce an enhanced event prediction having improved diagnostic confidence. For example, the respiratory acoustic event may include one or more of: a spontaneous cough, a cough subtype, a wheeze, a stridor, a crackle, an involuntary breath hold, an abnormal breathing pattern, a voluntary cough quality assessment, or a positional artifact.

As a second example, a server may include one or more processors and one or more computer-readable storage media storing instructions executable by the one or more processors to perform operations. The operations include receiving an audio stream from a patient and separating the audio stream into a speech stream and an artifact stream. The operations include processing, by a first artificial intelligence (AI) agent, the speech stream to conduct a conversation with the patient. The operations include performing an analysis of the artifact stream, by a second AI agent operating in parallel with the first AI agent, to detect a respiratory acoustic event. The operations include determining, by the second AI agent, an event prediction comprising an event indicator, an event type classification, an event duration, a confidence score, or a severity score for the respiratory acoustic event. The operations include applying, by a gating mechanism, one or more gating rules to the event prediction to determine an output action. Based on determining that the output action satisfies an escalation rule, the operations include escalating the conversation by: transferring the conversation to a medical professional, automatically initiating a call to emergency services, or both. Separating the audio stream into the speech stream and the artifact stream may include applying voice activity detection (VAD) to identify voiced speech segments in the audio stream, routing voiced speech segments to the speech stream, routing acoustic artifact segments to the artifact stream, and routing segments that include both voiced speech and acoustic artifacts to both the speech stream and the artifact stream. The second AI agent comprises a large language model (LLM) configured to analyze segments of the artifact stream and contextual conversational information and determine the event prediction comprising an event presence indicator, the event type classification, the event duration, the confidence score, and the severity score. For example, the event type classification may include at least one of: a spontaneous cough, a cough subtype, a wheeze, a stridor, a crackle, a rhonchus, an involuntary breath hold, an abnormal breathing pattern, or any combination thereof. The operations may include monitoring audio quality metrics of the artifact stream. For example, the audio quality metrics may include at least one of a signal-to-noise ratio, a frequency bandwidth, or a compression artifact level. The operations may include determining that the audio quality metrics are below an audio quality threshold and instructing the patient to switch to a higher-bandwidth communication medium to improve an audio quality of the artifact stream. The escalation rule may be based on at least one of: the event type classification satisfying a subtype-specific escalation criterion, a cumulative count of respiratory acoustic events within a time window exceeding an event count threshold, the severity score exceeding a severity threshold, or a combination of detected event subtypes satisfying a clinical pattern criterion. The operations may include maintaining a respiratory event log comprising, for each detected respiratory acoustic event, the event type classification, the confidence score, the severity score, an event timestamp, and the event duration. The operations may include outputting at least a portion of the respiratory event log in a form suitable for input to an electronic health records (EHR) platform. The operations may include receiving biometric data from a wearable device worn by the patient and correlating, by the second AI agent, the biometric data with the respiratory acoustic event to generate an enhanced event prediction.

As a third example, one or more non-transitory computer-readable storage media may store instructions executable by one or more processors to perform operations. The operations include receiving an audio stream from a patient and separating the audio stream into a speech stream and an artifact stream. The operations include processing, by a first artificial intelligence (AI) agent, the speech stream to conduct a conversation with the patient. The operations include performing an analysis of the artifact stream, by a second AI agent operating in parallel with the first AI agent, to detect a respiratory acoustic event. The operations include determining, by the second AI agent, an event prediction comprising an event indicator, an event type classification, an event duration, a confidence score, or a severity score for the respiratory acoustic event. The operations include applying, by a gating mechanism, one or more gating rules to the event prediction to determine an output action. Based on determining that the output action satisfies an escalation rule, the operations include escalating the conversation by: transferring the conversation to a medical professional, automatically initiating a call to emergency services, or both. The operations may include applying a temporal suppression rule that prevents the gating mechanism from triggering a response to a respiratory event that occurs within a suppression window following a prior trigger. The operations may include applying a cool down interval rule that enforces a minimum time interval between consecutive directed elicitation actions of a same type. The operations may include sending an elicitation instruction from the second AI agent to the first AI agent, where the elicitation instruction causes the first AI agent to include the elicitation instruction into the conversation to request the patient to perform a directed elicitation action. The operations may include analyzing audio produced by the patient during the directed elicitation action to produce an updated event prediction. For example, the directed elicitation action may include at least one of: requesting the patient to produce a voluntary cough, requesting the patient to speak a word or phrase selected to isolate respiratory acoustic artifacts in the artifact stream, requesting the patient to assume a physical position to alter a distribution of fluid in the patient; requesting the patient to reposition a device used by the patient to improve microphone proximity to a particular area of the patient, requesting the patient to move to a new location having less ambient noise than a current location of the patient, requesting the patient to switch to a higher-bandwidth communication medium, or any combination thereof. The operations may include maintaining a respiratory event log comprising, for each respiratory acoustic event, the event type classification, the confidence score, the severity score, an event timestamp, and the event duration, where the respiratory acoustic event comprises one or more of: a spontaneous cough, a cough subtype, a wheeze, a stridor, a crackle, an involuntary breath hold, an abnormal breathing pattern, a voluntary cough quality assessment, or a positional artifact. The operations may include receiving, by the second AI agent, contextual conversational information from the first AI agent. The contextual conversational information may include at least one of a conversation transcript, a conversation summary, or an electronic health record data of the patient. The second AI agent may include the contextual conversational information in the analysis of the artifact stream to detect the respiratory acoustic event.

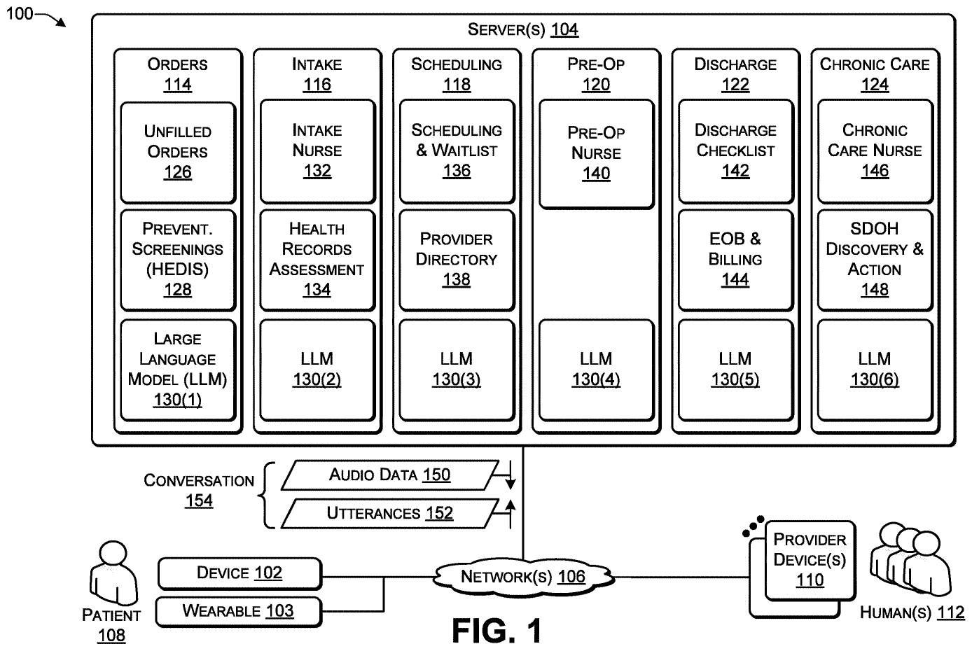

FIG. 1 is a block diagram of a system 100 illustrating large language models (LLMs) to perform specialized healthcare-related functions (e.g., roles), according to some implementations. The system 100 includes a device 102 connected to one or more servers 104 via one or more networks 106. The device 102 may be, for example, a smart phone, or another type of user device associated with a human 108. For example, the human 108 may be a current patient, a past patient, or a potential (future) patient. In some cases, the device 102 may be linked (“paired”) with a monitoring device (“monitor”) 103, such as a smart watch, a continuous glucose monitor (CGM), or another type of device that is capable of providing biometric readings associated with the human 108 to the device 102.

One or more provider devices 110 may be connected to the network 106. The provider devices 110 may be used by one or more medical professionals 112 (e.g., medical technicians, nurses, nurse practitioners, doctors, and the like) associated with a medical provider.

The server 104 may host multiple AI-based virtual assistants, such as the large language model (LLM) 130 virtual assistants. The virtual assistants LLM 130(1) to 130(6) shown in FIG. 1 each perform a particular healthcare-related role. Six LLM 130 are shown purely for illustration purposes. It should be understood that the number of virtual assistants LLM 130 may vary depending on how the healthcare functions (roles) are divided. The division of the healthcare functions influences the algorithm design, training data, and other details associated with the virtual assistants 130. A different division of the healthcare functions may result in fewer or more than six virtual assistants LLM 130. For illustration purposes, the six functions (roles) include orders 114, intake 116, scheduling 118, pre-op 120, discharge 122, and chronic care 124.

The orders 114 role may include the LLM 130(1) engaging in a conversation 154 with the human 108 to resolve unfilled orders 126. Unfilled orders 126 refers to prescribed medications that the human 108 has not picked up, prescribed medical procedures or follow-up appointments that the human 108 has not scheduled, and so on. The orders 114 role may include the LLM 130(1) entering into a conversation 154 with the human 108 to perform preventative screenings 128, including gathering data relative to Healthcare Effectiveness Data and Information Set (HEDIS) calculations.

The conversation 154 may include audio data 150 output by one of the LLM 130 and one or more utterances 152 from the human 108. The human 108 may speak the utterances 152 into a microphone of the device 102 and the device 102 may send the utterances 152 to the LLM 130 over the network 106. The LLM 130 may provide audio data 150 over the network 106 for playback by the device 102 to the human 108. The conversation 154 may be initiated by the LLM 130 or by the human 108.

The intake 116 role may include the LLM 130(2) engaging in the conversation 154 to perform the role of an intake nurse 132. For example, the conversation 154 may include the LLM 130(2) providing the human 108 with appointment-related information, such as a location and date and time associated with the appointment. The intake 116 role may include the LLM 130(2) engaging in the conversation 154 to perform a Health Records Assessment (HRA), determine a Risk Adjustment Factor (RAF), or the like.

The scheduling 118 role may include the LLM 130(3) engaging in the conversation 154 with the human 108 to perform scheduling and waitlisting related actions. For example, the LLM 130(3) may schedule the human 108 for an appointment and/or waitlist the human 108 for a consultation, surgery, or another type of appointment. The scheduling 118 role may include the LLM 130(3) engaging in the conversation 154 with the human 108 to access a provider directory to select a provider for the human 108. For example, if the human 108 has been asked (e.g., by a primary care physician) to have a consultation with a specialist, then the LLM 130(3) may identify a particular specialist in the provider directory 138 and schedule the human 108's consultation with the particular specialist.

The pre-op 120 role may include the LLM 130(4) engaging in the conversation 154 and taking on the role of a pre-op nurse 140 to go through a pre-op checklist with the human 108. For example, the pre-op checklist may include various pre-op do's and don'ts, such as what to eat, what not to eat (e.g., avoid solid foods at least 24 hours prior to a colonoscopy, avoid eating 12 hours prior to a blood glucose test, etc.), what medications to take, what medications to avoid taking (e.g., no beta blockers prior to a treadmill stress test), type of clothing to wear (e.g., loose clothing prior to a treadmill stress test), and so on.

The discharge 122 role may include the LLM 130(5) engaging in the conversation 154 to go over a discharge checklist 142. For example, the discharge checklist 142 may include describing what to do and what not do (e.g., don't perform certain action for a particular period of time after certain types of surgery), reviewing medications to take (e.g., take X every day, take Y as needed, and the like), highlighting warning signs (e.g., call medical provider immediately if the human 108 experiences particular symptoms, such as shortness of breath, dizziness, blurry vision, or the like), making follow-up appointments, and the like. The discharge 122 role may include the LLM 130(5) providing information regarding insurance coverage, explanation of benefits (EOB), and other billing-related information (e.g., amount that insurance should cover, amount that the human is responsible for paying, and so on).

The chronic care 124 role may include the LLM 130(6) performing the role of a chronic care nurse when engaging in the conversation 154 with the human 108. For example, the chronic care 124 role may include reviewing medical records and test results, and providing information regarding self-management, such as diet/nutrition suggestions, exercise suggestions, advice on managing prescription regimens, and information regarding monitoring symptoms. The chronic care 124 role may include the LLM 130(6) engaging in the conversation 154 to determine social determinants of health (SDOH), which are the non-medical factors that influence health outcomes. SDOH include the conditions in which people are born, grow, work, live, and age, and the systems shaping the conditions of daily life, such as economic policies, development agendas, social norms, social policies, and political systems.

Thus, a medical provider may deploy AI engines, in the form of a large language model (LLM) or similar AI, to perform specialized health-related tasks. Each AI engine may be designed and trained for a particular purpose, such as screening, intake, scheduling, pre-op, discharge, chronic care, and the like. The advantages of using AI engines include (i) reducing costs compared to using a human, (ii) freeing up humans to perform in-person functions (roles), (iii) performing the health-related tasks at times that are convenient, including outside regular business hours, and (iv) reducing human error when performing the health-related tasks.

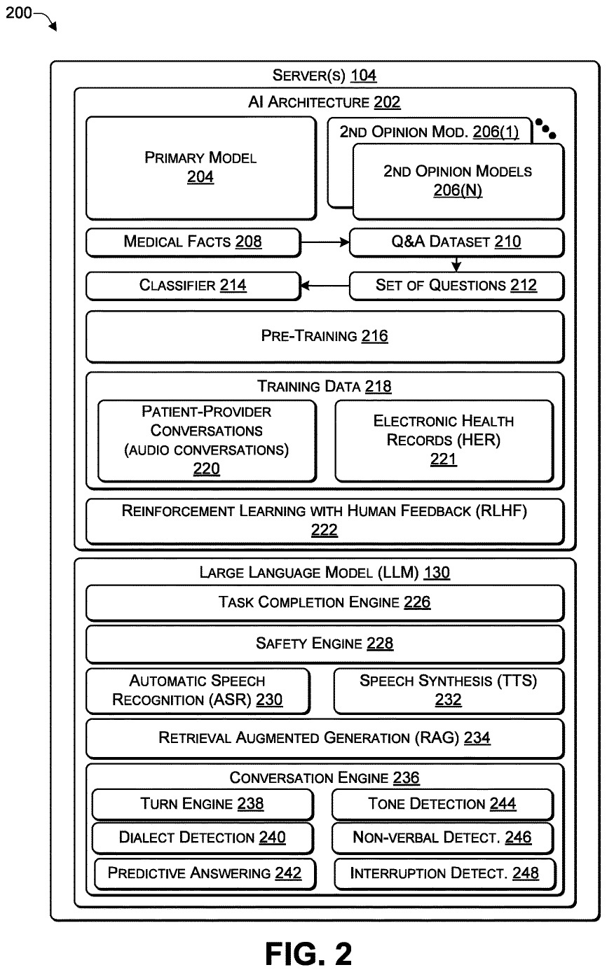

FIG. 2 is a block diagram 200 illustrating an artificial intelligence (AI) architecture 202, according to some implementations. The AI architecture 202 may be used to implement one or more of the LLMs (e.g., LLM 130(1), 130(2), 130(3), 130(4), 130(5), and 130(6) of FIG. 1) described herein.

The AI architecture 202 may include a primary model 204 which implements a primary role, such as screening, intake, scheduling, pre-op, discharge, chronic care, or any combination thereof. One or more second opinion models 206(1) to 206(N) (N>0) may be associated with the primary model 204. The second opinion models 206 are AI models, such as LLMs, that are trained to perform a more in-depth analysis than the LLM 130. Based on information extracted from the conversation 154 (of FIG. 1), one of the second opinion models 206 may perform a detailed (e.g., lengthy) analysis while the human 108 is engaged in the conversation 154 with one of the LLM 130. For example, the primary model 204 may have a 50 millisecond (ms) latency while the individual second opinion models 206 may have a 200 ms latency. If the second opinion model 206 determines that particular information provided by the LLM 130 during the conversation 154 can be clarified or expanded upon, then the second opinion model 206 may provide, to the LLM 130, the particular information along with a suggestion regarding how to present the particular information to the human 108. In this way, the primary model 204 and the second opinion models 206 are part of a low-latency conversational artificial intelligence (AI) architecture with a parallelized in-depth analysis feedback loop for healthcare-related applications. The purpose of reducing latency is to prevent the human from hanging up in frustration or due to the human erroneously concluding that the conversation has concluded or the virtual assistant has encountered an error.

A large corpus of medical facts 208 are gathered and converted into a Question & Answer (Q&A) dataset 210. The Q&A dataset 210 may be used to create a set of questions 212. For example, the medical facts 208, such as a description of an illness from a textbook, and converting the description into a Q&A structure, including “What is <illness>?”, “What are the symptoms of <illness>?”, “How is <illness>treated?”, “Who usually gets <illness>?”, “Is <illness>common?”, and so on. Similarly, tables and other structured data included in the medical facts 208 are converted into a Q&A structure. One or more additional LLMs may be used to determine whether a particular question in the set of questions 212 is relevant. A classifier 214 (e.g., a support vector machine (SVM) or another type of classifier) is used to determine (predict) a distance between individual questions and their corresponding answer to determine if the answer is correct. In pre-training 216, a question is a prompt (e.g., a query presented to the models 204, 206) and the answer is the response predicted by the models 204, 206. The Q&A dataset 210 may be used to fine tune the LLM 130. For example, instruction tuning may be used to further train the LLM 130 on the Q&A dataset 210 (e.g., that is in the form of (instruction, output) pairs), in a supervised fashion.

Training data 218 includes human-provider (e.g., patient-medical professional) conversations 220 in the form of audio data and electronic healthcare records (EHR) 221. By training the LLM 130 using conversation data, the LLM 130 is more suited to engaging in the conversation 154 with the human 108. The AI architecture 202 includes providing the LLM 130 with reinforcement learning with human feedback (RLHF) in which the LLM 130 is trained using a reward function (“reward model”) based on human feedback. The reward function provides the LLM 130 with a high reward for good output and a low reward for bad (poor) output. In this way, the LLM 130 is trained to provide the appropriate response at each turn in a conversation.

The LLM 130 (representing one of the LLMs 130(1) to 130(6) of FIG. 1) includes a task completion engine 226 that is tasked with completing a checklist during a particular conversation with the human. The task completion engine 226 enables the LLM 130 to complete tasks included in the checklist even while pursuing peripheral matters during the conversation, where such peripheral matters may help to build rapport between the virtual assistant and the human. For example, during the conversation with the human, the human may ask a question, such as a request for clarification of a particular topic (“Can I eat X prior to having the procedure?”). In response, the LLM 130 may provide information to answer the human's question. The LLM may also engage in banter about favorite foods, or the human's experiences with the foods, etc. After the LLM 130 answers the human's question and or engages in banter, the task completion engine 226 causes the LLM 130 to resume addressing items in the checklist. The LLM 130 may include a safety engine 228 to determine a medical accuracy of the information provided by the LLM 130 to the human. The safety engine 228 may determine when to perform a “kick out” and transfer a conversation between a human and an LLM to a medical professional.

The LLM 130 includes an automatic speech recognition module (ASR) 230 to perform speech recognition of the utterances 152 provided by the human 108. The LLM 130 includes a speech synthesis module 232 to perform text-to-speech (TTS) conversion. For example, after the LLM 130 looks up text-based information, the information is provided to the human in the form of speech using the speech synthesis module 232.

The LLM 130 includes a retrieval augmented generation (RAG) module 234. The RAG 234 is an AI framework to retrieve facts from an external knowledge base to provide the LLM 130 accurate and up-to-date information. The RAG 234 grounds the LLM 130 on external sources of knowledge to supplement the LLM 130. Implementing RAG 234 in an LLM-based question answering system enables the LLM 130 to access current, reliable facts, and the access to the sources of the LLM 130, enables output of the LLM 130 to be checked for accuracy. Using RAG, the AI-based virtual assistant described herein can provide accurate answers to a large number of questions that a human could not, such as menu options for a large number of local restaurants, details for a large number of insurance policies, details about a large number of hospital facilities (such as parking information, department locations, etc.).

The LLM 130 includes the conversation engine 236. The conversation engine 236 includes a turn engine 238, dialect detection 240, predictive answering 242, tone detection 244, nonverbal detection 246, and interruption detection 248. The turn engine 238 determines when the human has completed a turn and the LLM 130 has a turn in the conversation. The dialect detection 240 detects a dialect in the utterances of the human and causes the LLM 130 to output audio data having the same (or similar) dialect. For example, the dialect detection 240 may detect the dialect based on a particular word or a particular phrase in the human's utterances. To illustrate, the particular word or the particular phrase may be a vernacular term used in a particular geographic region.

The predictive answering 242 may predict multiple responses that the human may utter based on what the LLM 130 previously provided to the human. For example, the LLM 130 may provide a particular output (audio data) during the LLM's turn and then listen to the human during the human's turn. The predictive answering 242 may predict (i) possible human responses and (ii) corresponding answers based at least in part on the particular output provided during the LLM's turn. After the human has completed providing utterances in the human's turn, the LLM 130 may determine which of the predicted human responses the human provided and select a corresponding predicted answer. In this way, the predictive answering 242 is able to reduce the latency when providing a response after the human's turn.

The tone detection 244 is able to detect that the tone and changes to the tone in the human's utterances during the conversation. For example, the tone detection 244 may be able to detect a tone of the human's utterances, correlate the tone with a particular mood, and adjust the audio data output by the LLM 130 accordingly. To illustrate, the tone of the human's utterances may be determined based on a pitch of the utterances, a volume of the utterances, a particular word in the utterances, a particular phrase in the utterances, or any combination thereof. If a particular tone is detected, such as that of anger or frustration, then the LLM 130 may adjust the audio data output accordingly, including changing the pitch of the audio data, changing a volume of the audio data, inserting a particular word or a particular phrase in the audio data, or any combination thereof. In some cases, if the tone of the human is regressing instead of progressing (e.g., the human has become more angry or more frustrated in a subsequent turn), then the LLM 130 may offer to transfer (“escalate”) the conversation to a human. The non-verbal detection 246 may detect the human coughing, sneezing, sighing, crying, or providing another nonverbal audio cue. Detecting one of the nonverbal audio cues may cause the LLM 130 to ask additional (e.g., follow-up) questions, such as “How long have you had that cough?” (in response to detecting a cough), “How long have you had nasal congestion?” (in response to detecting a sneeze or sniffling), “Are you feeling okay?” (in response to detecting a loud sigh or crying), or the like. The interruption detection 248 may detect when the human is attempting to interrupt the LLM 130 and cause the LLM 130 to yield its turn and provide the human with a turn to enable the human to interject additional utterances into the conversation.

Thus, an AI architecture may include a primary model and one or more second opinion models to provide a low-latency conversational AI with a parallelized in-depth analysis and feedback loop in which the primary model engages in a low latency conversation with the human while the second opinion model gathers additional data and provides feedback (e.g., clarification or expansion of something that the primary model said to the human). Unlike conventional conversational AI's, the training data includes human provider audio-based conversations in addition to text-based training data. The LLM is trained using RLHF to provide improved conversational abilities to the LLM. The LLM may use a task completion engine to avoid missing any items on a checklist of items to be covered with the human. The conversation engine used by the LLM may include a turn engine to determine when the human's turn has ended and the LLM's turn begins, dialect detection to understand and respond to the human in a local dialect, predictive answering to reduce latency by predicting possible answers while the human is talking, tone detection to detect emotional cues (e.g., based on a change in pitch, a change in volume, or the like) in the human's voice, non-verbal detection (e.g., coughing, sneezing, crying, or the like), and interruption detection to detect when the human is attempting to interrupt the LLM. In this way, the LLM is able to provide and engage in a conversation with a human in a manner similar to a human. The purpose of reducing latency is to prevent the human from hanging up in frustration or due to the human erroneously concluding that the conversation has concluded or the virtual assistant has encountered an error.

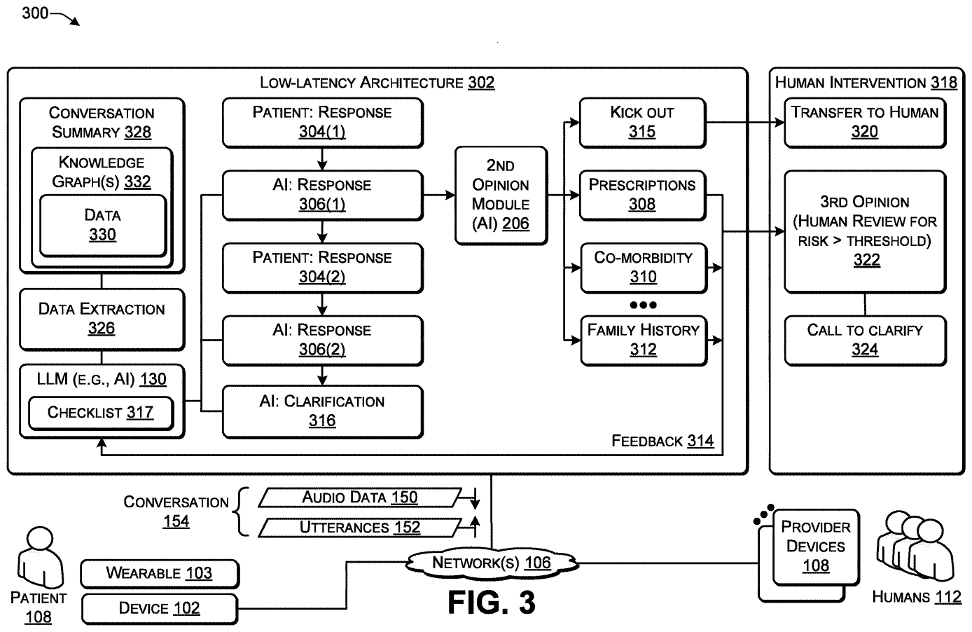

FIG. 3 is a block diagram of a system 300 that includes a low-latency conversational artificial intelligence (AI) architecture with a parallelized in-depth analysis and feedback loop, according to some implementations. The system 300 includes a low latency architecture 302 in which the LLM 130 receives a (first) response 304(1) from the human 108. The LLM 130 may engage in the conversation 154 with the human 108 based on a checklist 317 that identifies tasks that the LLM 130 is to complete during the conversation 154. The LLM 130 provides a (first) response 306(1) to the human 108. The LLM 130 receives a (second) response 304(2) from the human 108 and provides a (second) response 306(2) to the human 108. While the LLM 130 is engaged in the conversation 154 with the human 108, the second opinion module 206 may, in parallel (substantially at the same time), do a lengthier “deep dive” by performing research into the human response 304(1) and the AI response 306(1). For example, the research may include prescriptions 308, co-morbidity 310, family history 312 and the like. The prescriptions 308 may include determining whether the human 108 is being prescribed prescriptions appropriate to the human's conditions, determining whether the human 108 is being prescribed the correct dosages of the prescriptions, determining whether the prescribed prescriptions have any undesirable interactions or contraindications, and other prescription related information. The prescriptions 308 may include a dosage engine that is invoked when there is a mention of a drug, dosage, or both. The co-morbidity 310 may include determining whether the human 108 has two or more medical conditions that may be related to an underlying cause or that have an undesirable interaction. The family history 312 may include determining whether the human's parents, siblings, or other relatives have a history of medical conditions similar to that of the human 108.

Based on determining the research into the prescriptions 308, the co-morbidity 310, and the family history 312, the second opinion module 206 may determine whether the AI response 306(1) was appropriate or whether the AI response 306(1) may be clarified. If the second opinion module 206 determines that the response 306(1) is appropriate, the second opinion module 206 does not take further action. If the second opinion module 206 determines that the response 306(1) may be clarified, then the second opinion module 206 may provide feedback 314 to the LLM 130, causing the LLM 130 to provide a clarification 316. For example, the human response 304(1) may be “Can I have soup?” and the AI response 306(1) may be “yes, you can have soup.”. The clarification 316 may be “I previously said you could have soup. I noticed that either you or at least one of your family members have high blood pressure, so please check the sodium content of any pre-packaged soups and avoid soups with high sodium.” In this example, “you . . . have high blood pressure” was determined by checking co-morbidity 310 and “at least one of your family members have high blood pressure” was determined by checking family history 312. As another example, the clarification 316 may be “I noticed that you are currently taking <1st prescription> in the morning. Instead of taking <2nd prescription> in the morning as I previously suggested, I suggest taking <2nd prescription> in the evening, around 12 hours after taking <1st prescription>.” In this example, by checking the prescription 308, the second opinion module 206 may determine that the patient is taking the 1st prescription and that the 2nd prescription (mentioned in the AI response 306(1)) is not to be taken at the same time as the 1st prescription and instruct the LLM 130 to clarify when to take the 2nd prescription. As a further example, the clarification 316 may be “I noticed that your potassium was high in your most recent blood work. While I had previously suggested eating more avocados (for the oil), I suggest not having more than one avocado a week to avoid raising your potassium further, as avocados are high in potassium”. In this example, by checking the co-morbidity 310 that includes test results (such as the results of a blood workup, e.g., comprehensive metabolic panel), the second opinion module 206 may determine that the patient has a high potassium level and that avocados are high in potassium and clarify how many avocados the human can have in a particular time frame.

In some cases, the second opinion module 206 may determine that providing the clarification 316 is insufficient and that the conversation 154 would benefit from human intervention 318. In such cases, the second opinion module 206 may use a kick out 315 engine to determine to transfer the conversation 154 to the medical professional 320 (e.g., human nurse or doctor). In addition, if the second opinion module 206 determines that a risk to the human 108 is greater than a predetermined threshold, then the second opinion module 206 may initiate a request for a third opinion 322 in which a human (e.g., a doctor, such as a specialist) reviews the human's case and takes appropriate action (e.g., provides a call (to the human 108) to clarify 324 the situation). The kick out engine will be invoked for qualitative statements that suggest symptoms that are to be further evaluated.

While the LLM 130 is engaged in the conversation 154 (e.g., the responses 304, 306), one or more background processes may perform data extraction 326 to extract data 330 (e.g., pertinent information, such as facts) to create a conversation summary 328 which is stored in the form of a knowledge graph 332 (or another type of data structure). The conversation summary 328 and the data 330 may enable the LLM 130 to quickly access earlier information in a lengthy conversation. With medical-related AI, conversations may be lengthy and involve many turns (e.g., 50 to 100 turns is common and the conversation may, in some cases, include 200 or even 300 turns). In such lengthy conversations, the human 108 may reference previously provided information (“referenced information”) in a current response. If the referenced information was provided early in the conversation, a conventional virtual assistant may have “forgotten” the referenced information and thus provide responses that are frustrating to the human. Alternatively, a conventional virtual assistant may have to scan through a transcript of the conversation to determine the referenced information. Such a scan may cause a lengthy delay and introduce latency into the conversation 154. By performing data extraction 326 and creating the conversation summary 328 and maintaining the knowledge graph 332 storing the data 330, the LLM 130 is able to quickly access the information (data 330) referenced by the human, thereby reducing latency. The purpose of reducing latency is to provide a natural sounding conversation and prevent the human from hanging up (i) in frustration or (ii) due to the human erroneously concluding that the conversation has concluded or the virtual assistant has encountered an error.

Thus, a low latency architecture for a conversational AI includes an AI, such as an LLM, engaged in a conversation with the human. While the AI is engaged in the conversation with the human, a second opinion module does a more in-depth analysis of information provided by the human. If the second opinion module determines that a response provided by the conversational AI is to be clarified, the second opinion module provides feedback that the conversational AI uses to provide a clarification to the human during the conversation. By parallelizing the second opinion module with the conversational AI, latency is reduced, thereby making the conversation more natural. In addition, while the conversational AI is engaged in the conversation with the human, a data extraction module (in parallel) extracts facts from each human response and creates a conversation summary in the form of a data structure that can be quickly searched, such as a knowledge graph. The conversational AI can quickly access the conversation summary to look up information previously provided by the human that the human is referencing in a current turn in the conversation. The access to the conversation summary enables the conversational AI to reduce latency because the conversation AI is not pausing the conversation to perform a search of a transcript of the conversation.

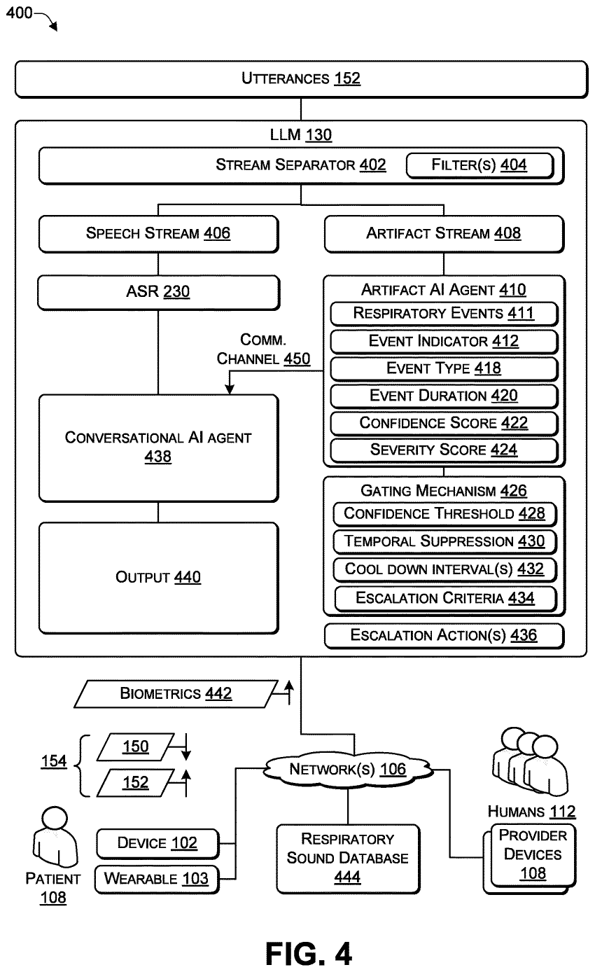

FIG. 4 is a block diagram of a system 400 in which, during a conversation between a patient 108 and an artificial intelligence (AI) agent, respiratory artifacts in an audio stream of the conversation are detected and processed in real time, according to some implementations.

The system 400 receives utterances 152 from the patient 108 via the device 102. In some cases (e.g., if available), the wearable 103 may provide biometrics 442. The utterances 152 are provided to the LLM 130, which may include a stream separator 402 and one or more filters 404. The stream separator 402 may use the filters 404 to separate the incoming audio into two parallel processing channels: (1) a speech stream 406 and (2) an artifact stream 408. In some cases, the filters 404 may apply signal processing techniques, including voice activity detection (VAD), spectral filtering, and/or noise suppression, to facilitate this separation. In other cases, the filters 404 may be AI-based filters, such as a first AI trained to identify and extract speech in an audio stream, a second AI trained to identify and extract respiratory audio artifacts from an audio stream, or both. In some cases, a single AI may be trained to perform both speech recognition and respiratory artifact recognition. Voice-based audio is routed to the speech stream 406, non-voice artifacts are routed to the artifact stream 408, and audio segments including both voice and acoustic artifacts (e.g., respiratory sounds, coughs, wheezes, and the like) may be routed to both speech stream 406 and the artifact stream 408.

The speech stream 406 is processed by an automatic speech recognition (ASR) module 230. The ASR 230 transcribes the patient's spoken words and provides the transcription to the conversational AI agent 438. The conversational AI agent 438 uses the transcription to engage in the conversation 154 with the patient 108 by generating an output 440 (e.g., spoken audio data) for delivery to the patient 108 via the device 102.

The artifact stream 408 is processed in parallel by an artifact AI agent 410. The artifact AI agent 410 may be a specialized acoustic classifier configured to detect and characterize respiratory events 411 embedded in the audio (utterances 152). The artifact AI agent 410 analyzes the artifact stream 408 and generates event predictions comprising: an event indicator 412, which indicates the presence or absence of a respiratory acoustic event; an event type 418, which classifies the type of respiratory event detected (e.g., cough, wheeze, stridor, crackle, abnormal breathing pattern, or the like); an event duration 420, which characterizes a length of the detected event in the audio stream; a confidence score 422, which quantifies the artifact AI agent's certainty that a genuine respiratory event has occurred; and a severity score 424, which provides a clinical severity estimate associated with the detected event.

In some cases, the artifact AI agent 410 may compare detected respiratory sounds against a respiratory sound database 444 that is accessible via the network(s) 106. The respiratory sound database 444 contains reference audio patterns for known respiratory conditions and events, enabling the artifact AI agent 410 to improve classification accuracy through similarity-based retrieval and augmented generation (RAG) techniques.

The event prediction produced by the artifact AI agent 410 may be passed to a gating mechanism 426. The gating mechanism 426 may apply one or more rules to determine whether the detected event should trigger a response, update an internal state, or be suppressed. The gating mechanism 426 includes a confidence threshold 428 (e.g., one or more actions may be performed after determining that the confidence score 422 exceeds the confidence threshold 428, thereby preventing spurious or low-confidence detections from affecting the conversation 154. The confidence threshold 428 may take into account an audio volume (e.g., in decibels) of individual respiratory events, how frequently a same or similar respiratory event is occurring, a time period during which the same or similar respiratory events are occurring, a time interval between a respiratory event and a prior respiratory event, and so on. The gating mechanism 426 may include a temporal suppression rule 430, which may prevent the gating mechanism 426 from triggering an output action for a respiratory acoustic event that occurs within a suppression window following a prior trigger, thereby reducing repeated alerts for the same respiratory event. In some cases, the artifact AI agent 410 may send a request, via a communication channel 450, to the conversational AI agent 438 to ask the patient 108 to perform one or more actions (directed elicitations), such as “Please cough”, “Please breathe deeply”, “Please place your phone's microphone on your chest”, and so on. The gating mechanism 426 may include cool down interval(s) 432 associated with individual events (e.g., a first interval for a coughing event, a second interval for a wheezing event, and so on), and with consecutive directed elicitation actions to avoid overwhelming the patient with repeated identical requests. The gating mechanism 426 may include escalation criteria 434, which determine whether a detected event or pattern of events warrants escalation to a medical professional or emergency service, based on factors such as event subtype, cumulative event count within a time window, severity score, or clinical pattern matching.

Based on the output of the gating mechanism 426, one or more escalation action(s) 436 may be triggered. The escalation actions 436 may include alerting a medical professional (one of the humans 112) via provider devices 110, transferring the conversation from the conversational AI agent 438 to one of the humans 112 (e.g., a nurse or physician), or initiating emergency protocols (e.g., alerting 911 or other emergency services).

The conversational AI agent 438 may receive messages from the artifact AI agent 410 via the inter-agent communication channel 450 and may use the information in the messages to dynamically adapt conversational behavior. For example, the conversational AI agent 438 may incorporate directed elicitation requests into the ongoing conversation 154 with the patient 108, such as asking the patient to perform specific actions, such as cough voluntarily, repeat a phrase (e.g., “say AHHHH”, and so on), change position (e.g., “tilt your head”), change a location of the device 102 (e.g., “place your phone near your chest”), to enable the artifact AI agent 410 to obtain additional audio for further analysis.

If the patient 108 is wearing a wearable 103 (e.g., a smart watch, continuous glucose monitor, wellness tracker, or the like), the system 400 may request access to the wearable device to receive biometrics 442. The biometrics 442 may include heart rate, blood oxygen saturation (SpO2), respiratory rate, and other physiological measurements. The artifact AI agent 410 may correlate the biometrics 442 with the respiratory events detected in the artifact stream 408 to improve diagnostic confidence and clinical relevance of the event predictions.

While the stream is illustrated in FIG. 4 as being separated into 2 streams, in some cases, the LLM 130 may use the AI agents 410 and 438 to analyze an audio stream that includes the utterances 152, where the artifact AI agent 410 has been trained to recognize respiratory artifacts in the audio stream (while disregarding speech in the audio stream) and the conversational AI agent 438 has been trained to recognize and extract speech in the audio stream (while disregarding non-speech artifacts, such as respiratory artifacts, in the audio stream). In other cases, a single AI agent (e.g., the conversational AI agent 438) may be trained to recognize both (i) respiratory artifacts in the audio stream and (ii) speech in the audio stream.

Thus, the system 400 enables a conversational AI agent to simultaneously conduct a natural language dialogue with a patient and perform real-time acoustic monitoring of the patient's respiratory health. In some cases, a single AI agent may engage in conversation with the patient while simultaneously and in parallel analyze respiratory artifacts in the audio received from the patient. In other cases, the audio from the patient may be split into a speech stream and an artifact stream processed by two AI agents in parallel. A gating mechanism may be used to reduce spurious actions while enabling the system to escalate when clinically warranted.

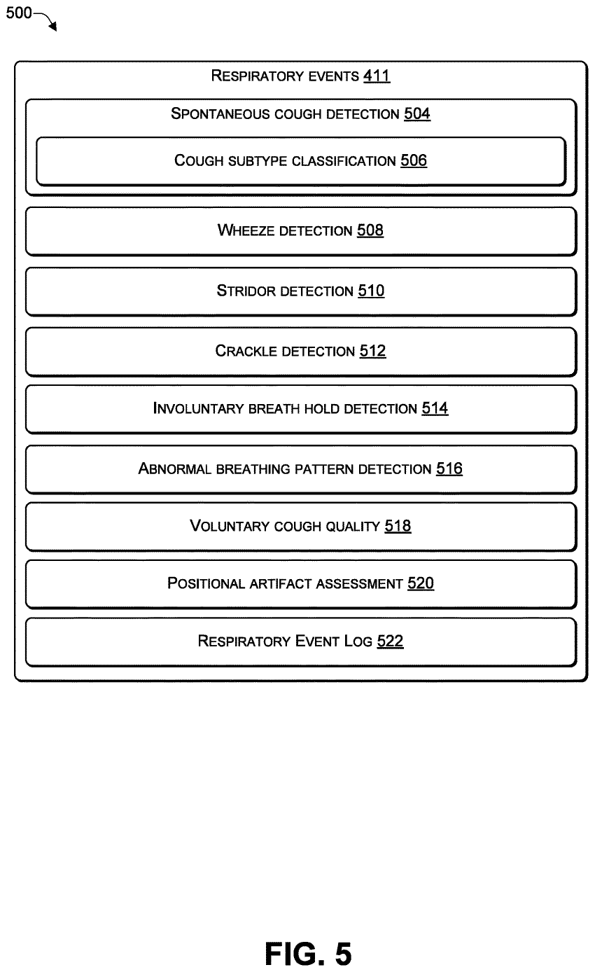

FIG. 5 is a block diagram 500 illustrating exemplary respiratory events 411 that may be detected by the artifact AI agent 410 or the conversational AI agent 438 of FIG. 4, according to some implementations. The respiratory events 411 represent a taxonomy of acoustic signals that may be present in the artifact stream 408 and that have diagnostic relevance for assessing a patient's respiratory health during a conversation.

Spontaneous cough detection 504 refers to the detection of an involuntary cough produced by the patient 108 during the conversation, without prompting by the conversational AI agent 438. Spontaneous coughs may indicate acute respiratory tract irritation, infection, airway inflammation, or other conditions. The artifact AI agent 410 distinguishes spontaneous coughs from incidental or conversational throat clearing based on acoustic features including onset sharpness, spectral energy distribution, and duration.

Cough subtype classification 506 refers to the further classification of a detected cough into clinically relevant subtypes. For example, cough subtypes may include dry cough (non-productive), wet or productive cough (suggesting mucus or fluid in the airways), barking cough (associated with croup or tracheal inflammation), rhonchus (a low-pitched, continuous, rattling lung sound that resembles snoring or gurgling that may occur when air flows through larger airways, such as the bronchi and trachea, that are partially blocked by mucus, secretions, or inflammation), whooping cough, and the like. The subtype classification informs the severity score 424 and may influence the escalation criteria 434.

Wheeze detection 508 refers to the detection of a high-pitched, continuous musical sound produced during breathing, typically associated with partial airway obstruction. Wheezing may be indicative of asthma, chronic obstructive pulmonary disease (COPD), bronchospasm, or other obstructive conditions. The artifact AI agent 410 characterizes wheeze events based on pitch, duration, phase of respiration (inspiratory versus expiratory), or any combination thereof.

Stridor detection 510 refers to the detection of a harsh, high-pitched breathing sound typically produced by turbulent airflow through a narrowed or partially obstructed upper airway. Stridor may indicate laryngeal or tracheal obstruction and is considered a potentially serious clinical finding that may trigger escalation criteria 434.

Crackle detection 512 refers to the detection of discontinuous, explosive sounds heard during respiration, also known as rales. Crackles may be fine (high-pitched, short duration) or coarse (lower-pitched, longer duration) and may be associated with pulmonary edema, pneumonia, interstitial lung disease, or fibrosis. The temporal and spectral characteristics of detected crackles are used to distinguish between fine and coarse subtypes.

Involuntary breath hold detection 514 refers to the detection of periods during which the patient 108 appears to involuntarily suspend breathing during the conversation. Involuntary breath holds may be associated with pain, anxiety, or respiratory distress and may signal a need for follow-up elicitation or escalation.

Abnormal breathing pattern detection 516 refers to the detection of irregular, rapid, shallow, or otherwise atypical breathing cadences that may be observable in the audio stream. Examples include tachypnea (abnormally rapid breathing), bradypnea (abnormally slow breathing), and irregular rhythm patterns such as Cheyne-Stokes or ataxic breathing.

Voluntary cough quality 518 refers to the assessment of the acoustic quality of a voluntary (elicited) cough produced by the patient 108 in response to a cough request from the conversational AI agent 438 of FIG. 4 (which may be received by the conversational AI agent 438 from the artifact AI agent 410). Voluntary cough quality metrics include cough strength, cough frequency (number of coughs per effort), and acoustic clarity, which may serve as indirect indicators of lung function and expiratory muscle strength.

Positional artifact assessment 520 refers to the detection and analysis of changes in respiratory acoustic signals following a positional elicitation request (e.g., the patient is asked to tilt their head, reposition their body, and so on). Changes in respiratory sounds following repositioning may help distinguish positional fluid accumulation (e.g., pleural effusion) from fixed structural abnormalities and may improve the artifact AI agent's diagnostic confidence.

After detecting one of the respiratory events 411, the artifact AI agent 410 of FIG. 4 may create a respiratory event log 522.

In some cases, the artifact AI agent 410 uses a respiratory sound database 444 (FIG. 4) in conjunction with trained classifiers to identify and categorize the respiratory events 411 described above. The structured outputs from this classification process are passed to the gating mechanism 426 (FIG. 4) for downstream processing and potential conversational integration.

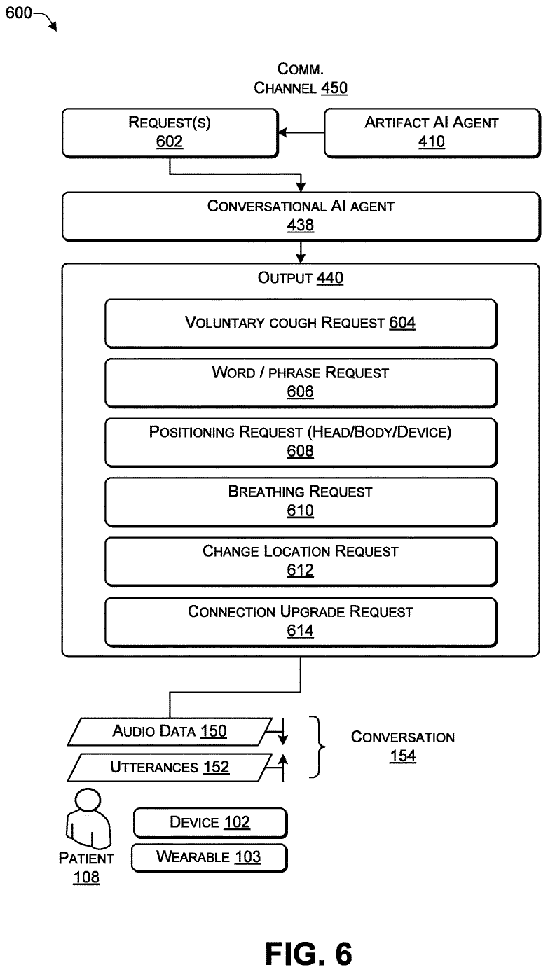

FIG. 6 is a block diagram 600 illustrating requests 602 that the conversational AI agent 438 may make to the patient 108 after the artifact AI agent 410 detects a respiratory artifact, according to some implementations.

When the artifact AI agent 410 detects a respiratory event 411 and the gating mechanism 426 determines that additional audio data may improve the accuracy or confidence of the event classification, the artifact AI agent 410 may transmit an elicitation request 602 to the conversational AI agent 438 via an inter-agent communication channel. The conversational AI agent 438 incorporates the elicitation request 602 into the ongoing conversation 154 as a directed elicitation request in the output 440, seamlessly embedding the request within the natural dialogue flow so that the patient experience is not disrupted.

Voluntary cough request 604: The conversational AI agent 438 asks the patient 108 to produce a deliberate cough (e.g., “Could you please cough for me?”). The artifact AI agent 410 analyzes the resulting voluntary cough to assess voluntary cough quality 518 (see FIG. 5), which can provide indirect information about lung function, expiratory muscle strength, and airway patency. The voluntary cough also serves as a controlled acoustic reference that can be compared against spontaneous cough detections 504 earlier (or later) in the conversation 154.

Word or phrase request 606: The conversational AI agent 438 asks the patient 108 to speak a specific word or phrase that is acoustically designed to elicit or isolate particular respiratory artifacts (e.g., “Please repeat the phrase ‘ninety-nine’ slowly.”, “Please say AHHHH”, “Please say EEEE”, and so on). Certain phonetic sequences produce characteristic resonance patterns that can reveal the presence of fluid consolidation in the airways or lungs. By analyzing the acoustic characteristics of the patient's spoken response, the artifact AI agent 410 can refine its classification of detected respiratory events.

Positioning request (head/body/device) 608: The conversational AI agent 438 may ask the patient 108 to assume a particular physical position or reposition the device 102 or wearable 103 (e.g., “Could you tilt your head slightly to the left and take a deep breath?” or “Please move your phone closer to your chest.”). Changes in respiratory sounds following repositioning facilitate positional artifact assessment 520 (see FIG. 5) and may help distinguish positional fluid accumulation from fixed structural abnormalities. Repositioning the microphone-bearing device 102 may also improve audio capture quality for the artifact AI agent 410.

Breathing request 610: The conversational AI agent 438 asks the patient 108 to perform a specific breathing maneuver (e.g., “Could you please take a slow, deep breath in and then breathe out fully?”). The controlled breathing maneuver produces an audio sample with known respiratory mechanics, enabling the artifact AI agent 410 to detect wheeze (508), stridor (510), crackle (512), or abnormal breathing patterns (516) under conditions that maximize acoustic contrast.

Change location request 612: The conversational AI agent 438 asks the patient 108 to move to a quieter location or to reduce background noise (e.g., “I'm having some difficulty hearing clearly. Could you move to a quieter room if possible?”). Environmental noise can significantly degrade the quality of the artifact stream 408 and reduce classification accuracy. By asking the patient to move to a quieter environment, the system reduces interference and improves the signal-to-noise ratio of subsequent respiratory audio analysis.

Connection upgrade request 614: The conversational AI agent 438 asks the patient 108 to switch to a higher-bandwidth communication medium (e.g., from a standard cellular call to a video call or a broadband audio platform). Lower-bandwidth codecs used in standard cellular telephony introduce audio compression artifacts that can mask or distort the high-frequency acoustic features characteristic of wheezes, crackles, and stridor. By requesting a higher-bandwidth connection, the system improves the frequency resolution available to the artifact AI agent 410, enabling more accurate respiratory event detection and subtype classification.

The requests 602 are incorporated into the conversation 154 as natural utterances delivered to the patient 108 via the output 440 of the conversational AI agent 438. The patient's responses are captured by the device 102 and/or wearable 103 and transmitted over the network(s) 106 to the LLM 130, where the audio is again processed by the stream separator 402, the artifact AI agent 410, and the gating mechanism 426, as described herein. In this way, the directed elicitation requests enable an iterative, dialogue-driven audio acquisition loop that progressively refines the artifact AI agent's respiratory event classifications while maintaining the conversational flow.

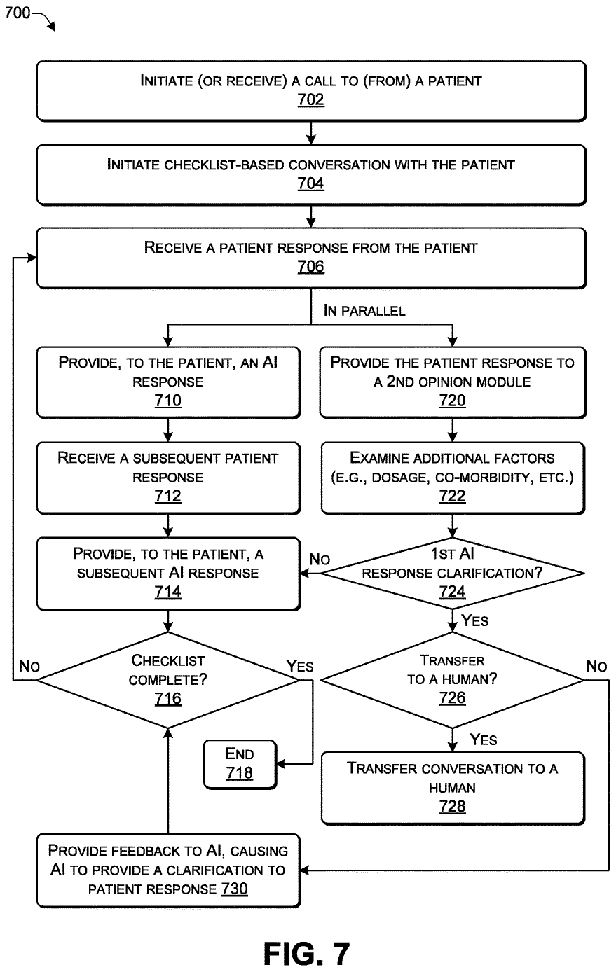

In the flow diagram of FIGS. 7, 8, 9, 11, 12, and 13 each block represents one or more operations that can be implemented in hardware, software, or a combination thereof. In the context of software, the blocks represent computer-executable instructions that, when executed by one or more processors, cause the processors to perform the recited operations. Generally, computer-executable instructions include routines, programs, objects, modules, components, data structures, and the like that perform particular functions or implement particular abstract data types. The order in which the blocks are described is not intended to be construed as a limitation, and any number of the described operations can be combined in any order and/or in parallel to implement the processes. For discussion purposes, the processes 700, 800, 900, 1100, 1200, and 1300 are described with reference to FIGS. 1, 2, 3, 4, 5, and 6 as described above, although other models, frameworks, systems and environments may be used to implement these processes.

FIG. 7 is a flowchart of a process 700 that includes analyzing a human response using a second opinion module, according to some implementations. The process 700 may be performed by one or more of the LLM's 130 of FIG. 1, 2, 3, or 4.

At 702, the process may initiate (or receive) a call to (from) a human. At 704, the process may initiate a checklist-based conversation with the human. At 706, the process may receive a human response from the human. For example, in FIG. 3, the LLM 130 may engage in the conversation 154 with the human 108. Either the LLM 130 or the human 108 may initiate a call prior to the conversation 154. The LLM 130 may use the checklist 317 to drive the conversation 154 with the human 108.

At 706, the process may receive a human response from the human. At 710, the process may provide, to the human, an AI response. At 712, the process may receive a subsequent human response. At 714, the process may provide, to the human, a subsequent AI response, and the process may proceed to 716. For example, in FIG. 3, the LLM 130 may receive the human response 304 from the human 108. The LLM 130 may provide, to the human 108, the AI response 306(1). The LLM 130 may receive a subsequent human response 304(2). The LLM 130 may provide, to the human 108, a subsequent AI response 306(2).

In parallel with 710, 712, and 714, a second opinion module (an AI) may, at 720, review the human response. At 722, the second opinion module may examine additional factors associated with the human (such as, dosage, co-morbidity, and the like). At 724, the second opinion module may determine whether the first AI response is to be clarified. If the second opinion module determines, at 724, that “no” the first AI response is not to be clarified, then the second opinion module may allow the process to continue conversing with the human. If the second opinion module determines, at 724, that “yes” the first AI response is to be clarified, then the second opinion module may determine, at 726, whether to transfer the conversation to a human. If the second opinion module determines, at 726, that “yes” the conversation is to be transferred to a human, then, at 728, the second opinion module may transfer the conversation to a human. If the second opinion module determines, at 726, that “no” the conversation is not to be transferred to a human, then the second opinion module may provide, at 730, feedback to the AI, causing the AI to provide a clarification (during the conversation) to the human response. The clarification may be to request additional information from the human for examination by the second opinion module. The second opinion module may then proceed to 716. For example, in FIG. 3, while the LLM 130 is engaged in the conversation 154 (e.g., responses 304(1), 306(1), 304(2), 306(2)), the second opinion module may examine the human response 304(1) in more depth/detail. For example, the second opinion module 206 may examine additional factors associated with the human 108, such as prescriptions (e.g., dosage, interactions, contraindications, or the like), co-morbidity 310, family history 312, and the like. After examining the additional factors, the second opinion module 206 may determine whether the AI response 306(1) is to be clarified. If the second opinion module 206 determines, that the AI response 306(1) is not to be clarified, then the second opinion module 206 allows the LLM 130 to continue the conversation 154 with the human 108. If the second opinion module 206 determines that the first AI response 306(1) is to be clarified, then the second opinion module 206 may determine whether to transfer the conversation 154 to a human. If the second opinion module 206 determines that the conversation 154 is to be transferred to a human, then the second opinion module may transfer the conversation 154 to one of the medical professionals 112. If the second opinion module 206 determines that the conversation 154 is not to be transferred to a human, then the second opinion module 206 may provide feedback 314 to the LLM 130, causing the LLM 130 to provide the clarification 316 during the conversation 154.

At 716, the process may determine whether a checklist is complete. If the process determines, at 716, that “yes” the checklist is complete, then the process may end the conversation. If the process determines, at 716 that “no” the checklist is incomplete, then the process may proceed back to 706 to receive a next human response from the human. For example, in FIG. 3, The LLM 130 may continue with the conversation 154 until the LLM 130 has completed the tasks in the checklist 317.

Thus, an AI, such as an LLM, may engage in a conversation with a human. The conversation may be initiated by the AI or by the human (e.g., human was asked to call a number that was routed to the AI). While the AI is engaged in the conversation with the human, a second opinion module does a more in-depth analysis of information provided by the human. If the second opinion module determines that a response provided by the conversational AI is to be clarified, the second opinion module provides feedback that the conversational AI uses to provide a clarification to the human during the conversation. By parallelizing the second opinion module with the conversational AI, latency is reduced, thereby making the conversation more natural while at the same time, enabling a second AI to perform a lengthier and more detailed analysis of information provided by the human.

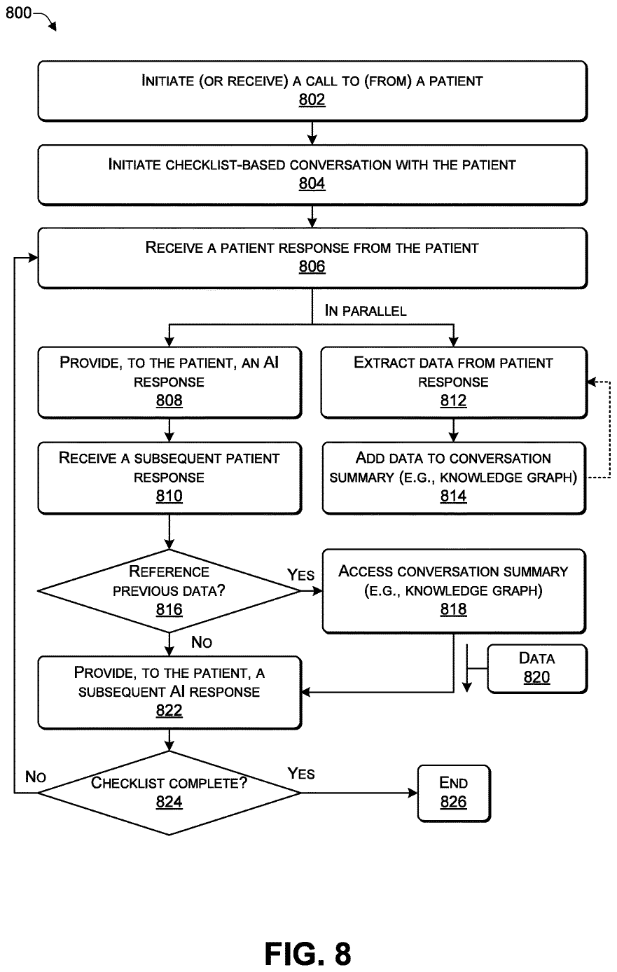

FIG. 8 is a flowchart of a process 800 that includes accessing a conversation summary, a knowledge graph, or both, according to some implementations. The process 800 may be performed by one or more of the LLM's 130 of FIGS. 1, 2, 3, and 4. While FIGS. 7 and 8 are shown as being separate flowcharts, it should be understood that the two flowcharts should be taken together, with 702, 704 and 706 corresponding to 802, 804 and 806, respectively. FIG. 8 illustrates the second opinion module operating in parallel with the primary LLM engaging in a conversation with the human. FIG. 8 illustrates creating a conversation summary in parallel with the primary LLM engaging in a conversation with the human.

At 802, the process may initiate (or receive) a call to (from) a human. At 804, the process may initiate a checklist-based conversation with the human. For example, in FIG. 3, the LLM 130 may engage in the conversation 154 with the human 108. Either the LLM 130 or the human 108 may initiate a call prior to the conversation 154. The LLM 130 may use the checklist 317 to drive the conversation 154 with the human 108.

At 806, the process may receive a human response from the human. At 808, the process may provide, to the human, an AI response. At 810, the process may receive a subsequent human response. At 816, the process may determine whether the subsequent human response referenced previously provided data. If the process determines, at 816, that “yes” the subsequent human response references previously provided data, then, at 818, the process may access a conversation summary. In some cases, the conversation summary may be stored in the form of a knowledge graph or another type of data structure. After accessing the conversation summary, the process may access data 820, and provide to the human, at 822, a subsequent AI response (based on accessing the data 820 in the conversation summary). At 824 the process may determine whether a checklist is complete. If the process determines, at 824, that “no” the checklist is not complete, then the process may proceed back to 806 to receive an additional human response. If the process determines, at 824, that “yes” the checklist is complete, then the process may end, at 826. For example, in FIG. 3, the LLM 130 may receive the human response 304(1) from the human 108. The LLM 130 may provide, to the human 108, the AI response 306(1). The LLM 130 may receive a subsequent human response 304(2). The LLM 130 may determine whether the subsequent human response 304(2) references previously provided data. If the LLM 130 determines that the subsequent human response 304(2) references previously provided data, then the LLM 130 may access the conversation summary 328. In some cases, the conversation summary 328 may be stored in the form of the knowledge graph 332 (or another type of data structure). After accessing the conversation summary 328, the LLM 130 may provide, to the human 108, a subsequent AI response 306(2), based on accessing the data in the conversation summary. The LLM 130 may determine whether the checklist 317 is complete. If the LLM 130 determines the checklist 317 is not complete, then the LLM 130 may receive an additional human response 304. If the LLM 130 determines the checklist 317 is complete, then the LLM 130 may end the conversation 154.

In parallel with 808 and 810, a data extraction module may, at 812, extract data (e.g., facts and other pertinent information) from the human response. At 814, the data extraction module may add the data to a conversation summary. For example, the conversation summary may be stored in the form of a knowledge graph or another type of data structure. The data extraction module may repeatedly monitor the human responses received at 806, extract data from the human response, at 812, and add the data to the conversation summary, at 814. In this way, the conversation summary is kept up to date. For example, in FIG. 3, while the LLM 130 is engaged in the conversation 154 with the human 108, the data extraction module 326 may extract data 330 (e.g., facts and other pertinent information) from the human response 304(1) and add the data 330 to the conversation summary 328. For example, the conversation summary 328 may be stored in the form of the knowledge graph 332 or another type of data structure. The data extraction module 326 may repeatedly monitor the human responses 304, extract data 330 from the human responses 304 and add the data 330 to the conversation summary 328. In this way, the conversation summary 328 is kept up to date.

Thus, a low latency architecture for a conversational AI includes an AI, such as an LLM, engaged in a conversation with the human. While the AI is engaged in the conversation with the human, a data extraction module (in parallel) extracts facts from each human response and creates a conversation summary in the form of a data structure that can be quickly searched, such as a knowledge graph. The conversational AI can quickly access the conversation summary to look up data that was previously provided by the human that the human is referencing in a current response (turn) in the conversation. The access to the conversation summary enables the conversational AI to reduce latency because the conversation AI is not pausing the conversation to perform a search of a transcript of the conversation.

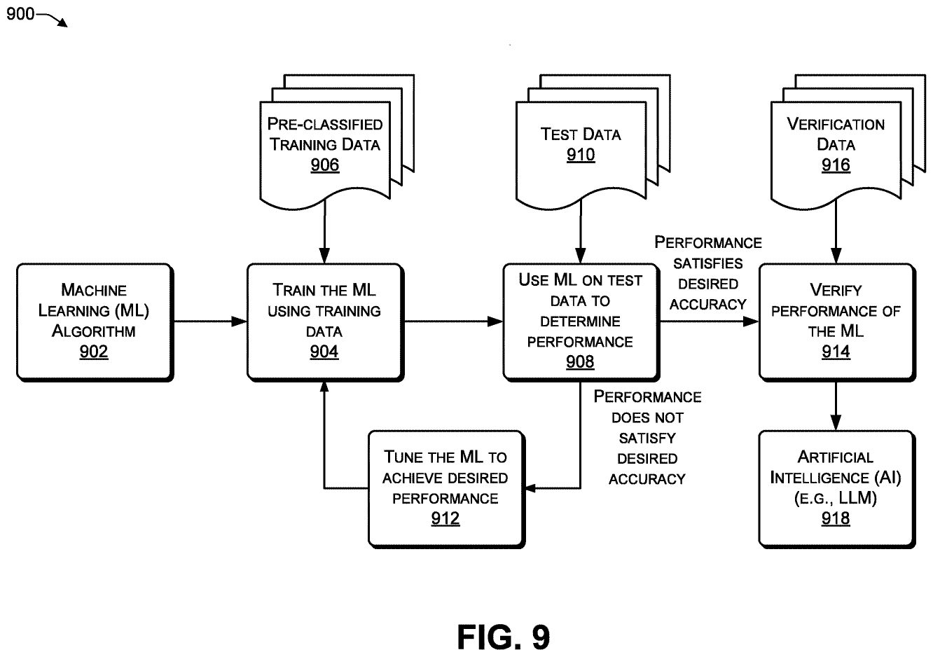

FIG. 9 is a flowchart of a process 900 to train a machine learning algorithm, according to some implementations. For example, the process 900 may be performed during the pre-training 216 or other training described herein.

At 902, a machine learning algorithm (e.g., software code) may be created by one or more software designers. For example, the LLMs 130 may be created by software designers. At 904, the machine learning algorithm may be trained using pre-classified training data 906. For example, the training data 906 may have been pre-classified by humans, by machine learning, or a combination of both. After the machine learning has been trained using the pre-classified training data 906, the machine learning may be tested, at 908, using test data 910 to determine a performance metric of the machine learning. The performance metric may include, for example, precision, recall, Frechet Inception Distance (FID), or a more complex performance metric. For example, in the case of a classifier, the accuracy of the classification may be determined using the test data 910.

If the performance metric of the machine learning does not satisfy a desired measurement (e.g., 95%, 98%, 99% in the case of accuracy), at 908, then the machine learning code may be tuned, at 912, to achieve the desired performance measurement. For example, at 912, the software designers may modify the machine learning software code to improve the performance of the machine learning algorithm. After the machine learning has been tuned, at 912, the machine learning may be retrained, at 904, using the pre-classified training data 906. In this way, 904, 908, 912 may be repeated until the performance of the machine learning is able to satisfy the desired performance metric. For example, in the case of a classifier, the classifier may be tuned to be able to classify the test data 910 with the desired accuracy.

After determining, at 908, that the performance of the machine learning satisfies the desired performance metric, the process may proceed to 914, where verification data 916 may be used to verify the performance of the machine learning. After the performance of the machine learning is verified, at 914, the machine learning 902, which has been trained to provide a particular level of performance may be used as an artificial intelligence (AI) 918, such as the LLM 130, the conversational AI agent 438, the artifact AI agent 408, and so on. For example, the conversational AI agent 438 may be trained using audio that includes speech. The artifact AI agent 408 may be trained using audio that includes respiratory artifacts.

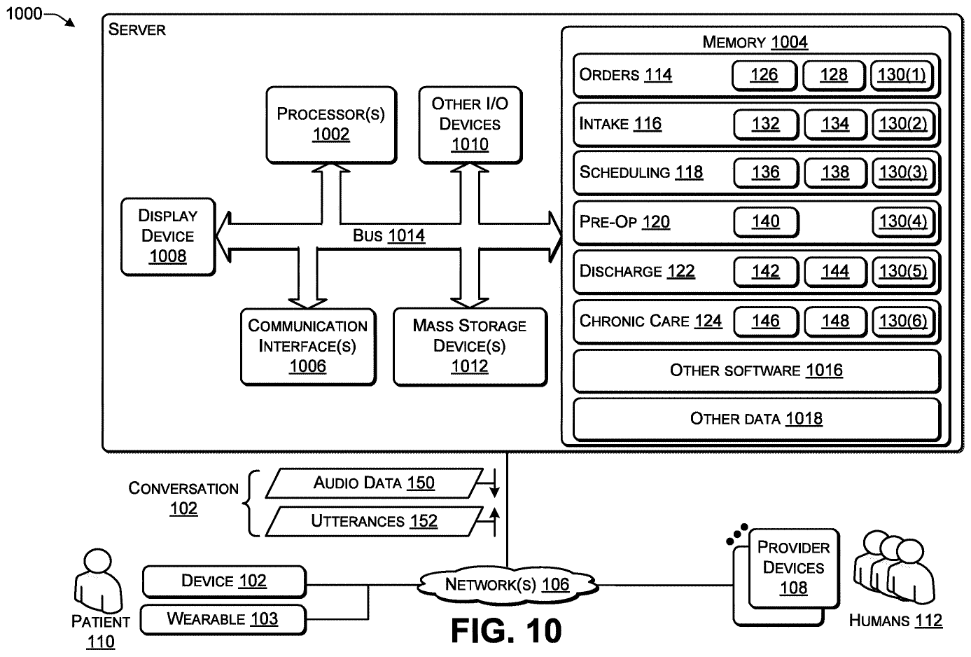

FIG. 10 illustrates an example configuration of a device 1000 that can be used to implement the systems and techniques described herein. For example, the device 1000 may be a server (or a set of servers) used to host one or more of the components described herein. In some cases, the systems and techniques described herein may be implemented as an application programming interface (API), a plugin, or another type of implementation.

The device 1000 may include one or more processors 1002 (e.g., central processing unit (CPU), graphics processing unit (GPU), or the like), a memory 1004, communication interfaces 1006, a display device 1008, other input/output (I/O) devices 1010 (e.g., keyboard, trackball, and the like), and one or more mass storage devices 1012 (e.g., disk drive, solid state disk drive, or the like), configured to communicate with each other, such as via one or more system buses 1014 or other suitable connections. While a single system bus 1014 is illustrated for ease of understanding, it should be understood that the system bus 1014 may include multiple buses, such as a memory device bus, a storage device bus (e.g., serial ATA (SATA) and the like), data buses (e.g., universal serial bus (USB) and the like), video signal buses (e.g., ThunderBolt®, digital video interface (DVI), high definition media interface (HDMI), and the like), power buses, etc.

The processors 1002 are one or more hardware devices that may include a single processing unit or a number of processing units, all of which may include single or multiple computing units or multiple cores. The processors 1002 may include a graphics processing unit (GPU) that is integrated into the CPU or the GPU may be a separate processor device from the CPU. The processors 1002 may be implemented as one or more microprocessors, microcomputers, microcontrollers, digital signal processors, central processing units, graphics processing units, state machines, logic circuitries, and/or any devices that manipulate signals based on operational instructions. Among other capabilities, the processors 1002 may be configured to fetch and execute computer-readable instructions stored in the memory 1004, mass storage devices 1012, or other computer-readable media.

Memory 1004 and mass storage devices 1012 are examples of computer storage media (e.g., memory storage devices) for storing instructions that can be executed by the processors 1002 to perform the various functions described herein. For example, memory 1004 may include both volatile memory and non-volatile memory (e.g., random access memory (RAM), read only memory (ROM), or the like) devices. Further, mass storage devices 1012 may include hard disk drives, solid-state drives, removable media, including external and removable drives, memory cards, flash memory, floppy disks, optical disks (e.g., compact disc (CD), digital versatile disc (DVD), a storage array, a network attached storage (NAS), a storage area network (SAN), or the like. Both memory 1004 and mass storage devices 1012 may be collectively referred to as memory or computer storage media herein and may be any type of non-transitory media capable of storing computer-readable, processor-executable program instructions as computer program code that can be executed by the processors 1002 as a particular machine configured for carrying out the operations and functions described in the implementations herein.

The device 1000 may include one or more communication interfaces 1006 for exchanging data via the network 106. The communication interfaces 1006 can facilitate communications within a wide variety of networks and protocol types, including wired networks (e.g., Ethernet, Data Over Cable Service Interface Specification (DOCSIS), digital subscriber line (DSL), Fiber, universal serial bus (USB) etc.) and wireless networks (e.g., wireless local area network (WLAN), global system for mobile (GSM), code division multiple access (CDMA), 802.11, Bluetooth, Wireless USB, ZigBee, cellular, satellite, etc.), the Internet and the like. Communication interfaces 1006 can also provide communication with external storage, such as a storage array, network attached storage, storage area network, cloud storage, or the like.

The display device 1008 may be used for displaying content (e.g., information and images) to users. Other I/O devices 1010 may be devices that receive various inputs from a user and provide various outputs to the user, and may include a keyboard, a touchpad, a mouse, a gaming controller (e.g., joystick, steering controller, accelerator pedal, brake pedal controller, virtual reality (VR) headset, VR glove, or the like), a printer, audio input/output devices, and so forth.

The computer storage media, such as memory 1004 and mass storage devices 1012, may be used to store any of the software and data described herein.

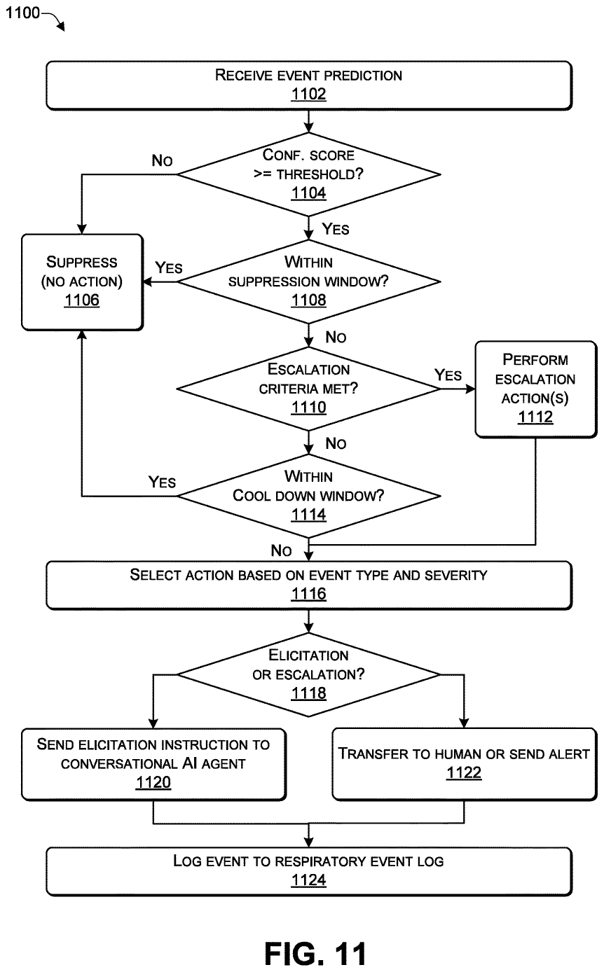

FIG. 11 is a flowchart of a process 1100 that applies the gating mechanism 426 to an event prediction, according to some implementations. The process 1100 may be performed by the LLM 130, and in particular by the gating mechanism 426 described with reference to FIG. 4.

At 1102, the process 1100 may receive an event prediction from the artifact AI agent 410. For example, in FIG. 4, the artifact AI agent 410 may analyze the artifact stream 408 and produce an event prediction comprising the event indicator 412, event type 418, event duration 420, confidence score 422, and severity score 424.

At 1104, the process 1100 may determine whether the confidence score 422 meets or exceeds the confidence threshold 428. If the process determines, at 1104, that “no” the confidence score 422 does not meet the confidence threshold 428, then the process may suppress the event and take no action, at 1106, and the process may end. If the process determines, at 1104, that “yes” the confidence score 422 meets or exceeds the confidence threshold 428, the process may proceed to 1108.

At 1108, the process 1100 may determine whether the detected event falls within a temporal suppression window 430 following a prior trigger. If the process determines, at 1108, that “yes” the event falls within the suppression window, the process may suppress the event and take no action, at 1106. If the process determines, at 1108, that “no” the event does not fall within the suppression window, the process may proceed to 1110.

At 1110, the process 1100 may determine whether the escalation criteria 434 are met. The escalation criteria 434 may be based on one or more of: the event type classification 418 satisfying a subtype-specific escalation criterion; a cumulative count of respiratory acoustic events within a time window exceeding an event count threshold; the severity score 424 exceeding a severity threshold; or a combination of detected event subtypes satisfying a clinical pattern criterion. If the process determines, at 1110, that “yes” the escalation criteria 434 are met, the process may perform one or more escalation actions 436, at 1112. For example, the escalation actions 436 may include transferring the conversation to a medical professional 112 or alerting one or more provider devices 110. After performing the escalation action(s), the process may proceed to 1124 to log the event. If the process determines, at 1110, that “no” the escalation criteria 434 are not met, the process may proceed to 1114.

At 1114, the process 1100 may determine whether the current event falls within a cooldown interval 432 associated with its event type. The cooldown interval 432 may enforce a minimum time interval between consecutive directed elicitation actions of the same type, to avoid overwhelming the patient 108 with repeated identical requests. If the process determines, at 1114, that “yes” the event falls within the cooldown interval, the process may suppress the event and take no action, at 1106. If the process determines, at 1114, that “no” the event does not fall within the cooldown interval, the process may proceed to 1116.

At 1116, the process 1100 may select an output action based on the event type 418 and the severity score 424. At 1118, the process 1100 may determine whether the selected output action is a directed elicitation action or an escalation action. If the selected output action is an elicitation action, the process may, at 1120, send an elicitation instruction to the conversational AI agent 438 via the communication channel 450. The conversational AI agent 438 may incorporate the elicitation instruction into the ongoing conversation 154 with the patient 108. If the selected output action is an escalation action, the process may, at 1122, transfer the conversation to a medical professional 112 or send an alert to one or more provider devices 110. In either case, the process may proceed to 1124 to log the event.

At 1124, the process 1100 may log the detected event to the respiratory event log 522. For each detected respiratory acoustic event, the respiratory event log 522 may store the event type classification 418, the confidence score 422, the severity score 424, an event timestamp, and the event duration 420. The respiratory event log 522 may subsequently be output in a form suitable for input to an electronic health records (EHR) platform, or provided to a medical professional 112 together with a conversation summary in the event of an escalation.

Thus, the gating mechanism 426 applies a sequence of suppression rules, including confidence threshold 428, temporal suppression 430, escalation criteria 434, and cooldown intervals 432, to determine the appropriate output action for each detected respiratory acoustic event, ensuring that clinically meaningful events result in directed elicitation requests or escalation actions, while spurious or repeated detections are suppressed.

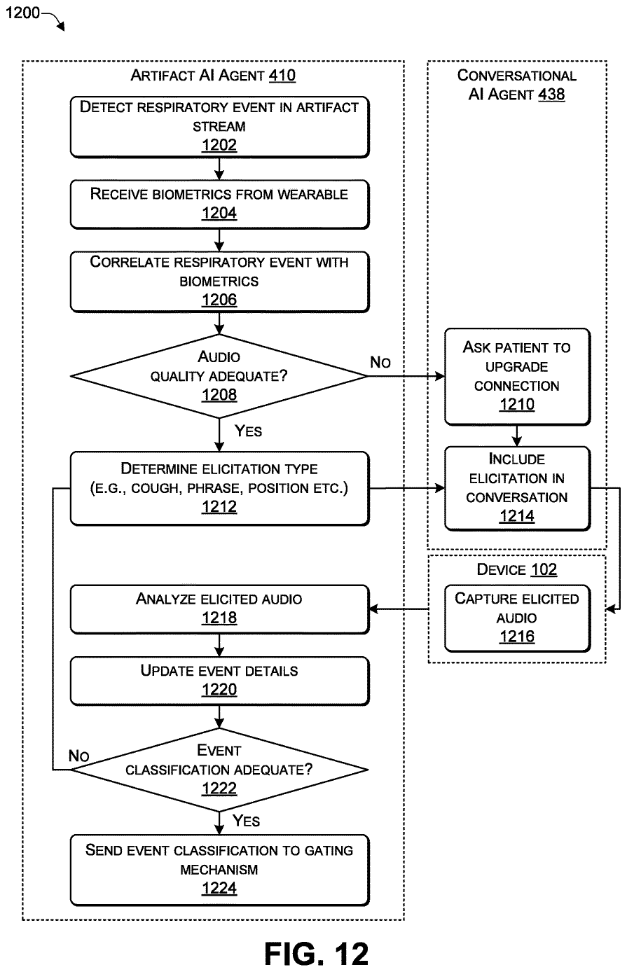

FIG. 12 is a flowchart of a process 1200 that includes the artifact AI agent 410 instructing the conversational AI agent 438 to perform directed elicitation actions, according to some implementations. The process 1200 may be performed by the LLM 130, and in particular by the artifact AI agent 410 and the conversational AI agent 438 operating in parallel via the communication channel 450 described with reference to FIGS. 4 and 6.

At 1202, the artifact AI agent 410 may detect a respiratory event in the artifact stream 408. For example, the artifact AI agent 410 may analyze the artifact stream 408 to identify one or more respiratory events 411, such as spontaneous cough detection 504, wheeze detection 508, stridor detection 510, crackle detection 512, involuntary breath hold detection 514, or abnormal breathing pattern detection 516, as described with reference to FIG. 5.

At 1204, if the patient 108 is wearing a wearable 103, the artifact AI agent 410 may receive biometrics 442 from the wearable 103. At 1206, the artifact AI agent 410 may correlate the detected respiratory event with the biometrics 442 to improve diagnostic confidence and the clinical relevance of the event prediction. For example, the biometrics 442 may include heart rate, blood oxygen saturation (SpO2), or respiratory rate, and correlating these measurements with the detected respiratory event may allow the artifact AI agent 410 to produce a more accurate severity score 424.

At 1208, the artifact AI agent 410 may determine whether the audio quality of the artifact stream 408 is adequate for reliable classification. The audio quality may be assessed based on one or more metrics, such as signal-to-noise ratio, frequency bandwidth, or compression artifact level. If the process determines, at 1208, that “no” the audio quality is not adequate, the process may, at 1210, cause the conversational AI agent 438 to ask the patient 108 to upgrade the connection, for example by switching from a standard cellular call to a higher-bandwidth communication medium (see connection upgrade request 614, FIG. 6). If the process determines, at 1208, that “yes” the audio quality is adequate, the process may proceed to 1212.

At 1212, the artifact AI agent 410 may determine the type of directed elicitation action most likely to yield audio data that improves classification accuracy. For example, the artifact AI agent 410 may determine that a voluntary cough request 604, a word or phrase request 606, a positioning request 608, a breathing request 610, or a change location request 612 is appropriate, as described with reference to FIG. 6. The artifact AI agent 410 may transmit the elicitation instruction to the conversational AI agent 438 via the communication channel 450.

At 1214, the conversational AI agent 438 may incorporate the elicitation instruction into the ongoing conversation 154 and deliver the request to the patient 108 as part of the output 440, so that the patient experience is not disrupted. At 1216, the device 102 and/or wearable 103 may capture the audio produced by the patient 108 in response to the directed elicitation action. The captured audio may be transmitted over the network(s) 106 to the LLM 130 and processed by the stream separator 402 and artifact AI agent 410.

At 1218, the artifact AI agent 410 may analyze the elicited audio in the artifact stream 408. At 1220, the artifact AI agent 410 may update the event details, including the event type 418, event duration 420, confidence score 422, and severity score 424, based on the analysis of the elicited audio. The artifact AI agent 410 may also compare the detected acoustic features against the respiratory sound database 444 using similarity-based retrieval to improve event type classification accuracy.

At 1222, the process 1200 may determine whether the event classification is adequate. If the process determines, at 1222, that “no” the classification is not adequate, the process may return to 1212 to determine a further elicitation type, initiating another iteration of the directed elicitation loop. If the process determines, at 1222, that “yes” the classification is adequate, the process may proceed to 1224.

At 1224, the artifact AI agent 410 may send the final event classification to the gating mechanism 426 (described with reference to FIG. 11) for downstream processing and determination of the output action.

Thus, the process 1200 enables a directed elicitation loop in which the artifact AI agent 410 and the conversational AI agent 438 cooperate via the communication channel 450 to iteratively acquire audio data from the patient 108 and refine respiratory event classifications, while maintaining the natural flow of the conversation 154.

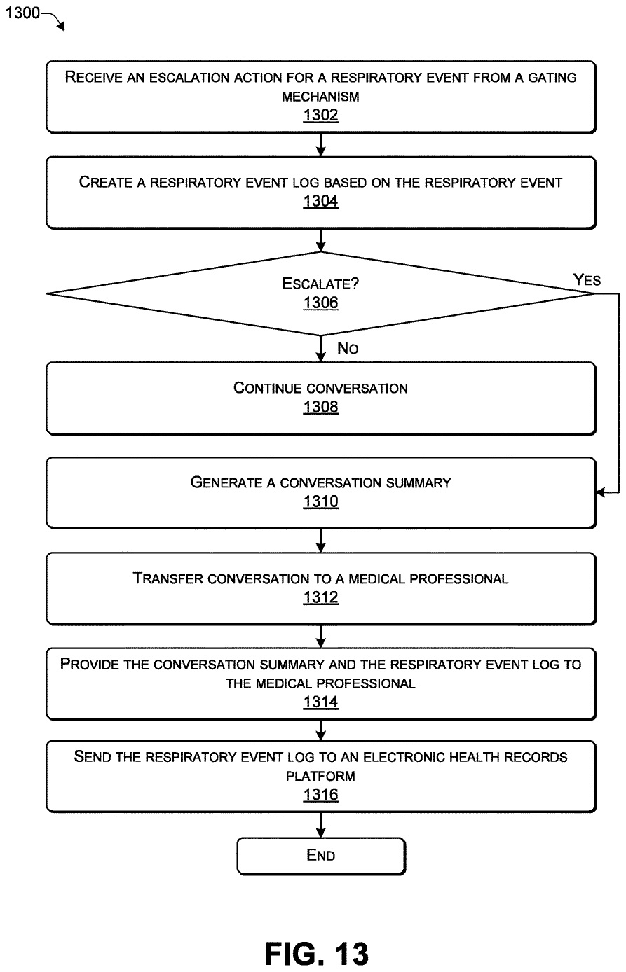

FIG. 13 is a flowchart of a process 1300 that includes determining whether to escalate a conversation to a medical professional, according to some implementations. The process 1300 may be performed by the LLM 130, and in particular by the gating mechanism 426 and the escalation actions 436 described with reference to FIGS. 4 and 11.

At 1302, the process 1300 may receive an escalation action for a respiratory event from the gating mechanism 426. For example, in FIG. 11, when the gating mechanism 426 determines that the escalation criteria 434 are met and the selected output action is an escalation action, the process may invoke the process 1300.

At 1304, the process 1300 may create a respiratory event log 522 based on the respiratory event. The respiratory event log 522 may include, for each detected respiratory acoustic event, the event type classification 418, the confidence score 422, the severity score 424, an event timestamp, and the event duration 420.

At 1306, the process 1300 may determine whether to escalate. If the process determines, at 1306, that “no” the conversation should not be escalated, the process may continue the conversation, at 1308, without transferring to a medical professional 112. If the process determines, at 1306, that “yes” the conversation should be escalated, the process may proceed to 1310.

At 1310, the process 1300 may generate a conversation summary. The conversation summary may include a transcript of the conversation 154, relevant data extracted from prior patient responses, and the respiratory event log 522 created at 1304. At 1312, the process 1300 may transfer the conversation 154 to a medical professional 112 via one or more provider devices 110. At 1314, the process 1300 may provide the conversation summary and the respiratory event log 522 to the medical professional 112, enabling the medical professional 112 to review the patient's respiratory history and the context of the conversation before intervening.

At 1316, the process 1300 may send the respiratory event log 522 to an electronic health records (EHR) platform, outputting at least a portion of the respiratory event log 522 in a form suitable for input to the EHR platform, so that the detected respiratory events are preserved in the patient's permanent medical record.

Thus, the process 1300 ensures that when a respiratory acoustic event meets the escalation criteria 434, the conversation 154 is transferred to a medical professional 112, who is provided with both the conversation summary and the respiratory event log 522, and that the respiratory event log 522 is preserved in the patient's EHR for future clinical reference.

The example systems and computing devices described herein are merely examples suitable for some implementations and are not intended to suggest any limitation as to the scope of use or functionality of the environments, architectures and frameworks that can implement the processes, components and features described herein. Thus, implementations herein are operational with numerous environments or architectures, and may be implemented in general purpose and special-purpose computing systems, or other devices having processing capability. Generally, any of the functions described with reference to the figures can be implemented using software, hardware (e.g., fixed logic circuitry) or a combination of these implementations. The term “module,” “mechanism” or “component” as used herein generally represents software, hardware, or a combination of software and hardware that can be configured to implement prescribed functions. For instance, in the case of a software implementation, the term “module,” “mechanism” or “component” can represent program code (and/or declarative-type instructions) that performs specified tasks or operations when executed on a processing device or devices (e.g., CPUs or processors). The program code can be stored in one or more computer-readable memory devices or other computer storage devices. Thus, the processes, components and modules described herein may be implemented by a computer program product.

Furthermore, this disclosure provides various example implementations, as described and as illustrated in the drawings. However, this disclosure is not limited to the implementations described and illustrated herein, but can extend to other implementations, as would be known or as would become known to those skilled in the art. Reference in the specification to “one implementation,” “this implementation,” “these implementations” or “some implementations” means that a particular feature, structure, or characteristic described is included in at least one implementation, and the appearances of these phrases in various places in the specification are not necessarily all referring to the same implementation.

Although the present technology disclosed has been described in connection with several implementations, the technology disclosed is not intended to be limited to the specific forms set forth herein. On the contrary, it is intended to cover such alternatives, modifications, and equivalents as can be reasonably included within the scope of the technology disclosed as defined by the appended claims.

1. A method, comprising:
receiving, by one or more processors, an audio stream from a patient;
separating, by the one or more processors, the audio stream into a speech stream and an artifact stream;
processing, by a first artificial intelligence (AI) agent, the speech stream to conduct a conversation with the patient;
performing an analysis of the artifact stream, by a second AI agent operating in parallel with the first AI agent, to detect a respiratory acoustic event;
determining, by the second AI agent, an event prediction comprising one or more of an event indicator, an event type classification, an event duration, a confidence score, or a severity score for the respiratory acoustic event;
applying, by a gating mechanism, one or more gating rules to the event prediction to determine an output action; and
based on determining that the output action satisfies an escalation rule, escalating the conversation by:
transferring the conversation to a medical professional;
automatically initiating a call to emergency services; or
both.

2. The method of claim 1, wherein applying the one or more gating rules comprises:
applying a temporal suppression rule that prevents the gating mechanism from triggering the output action within a suppression window following a prior trigger; and
applying a cooldown interval rule that enforces a minimum time interval between consecutive directed elicitation actions of a same type.

3. The method of claim 1, further comprising:
sending an elicitation instruction from the second AI agent to the first AI agent, that causes the first AI agent to perform a directed elicitation action by incorporating the elicitation instruction into the conversation; and
analyzing, by the second AI agent, audio produced by the patient in response to the directed elicitation action to produce an updated event prediction.

4. The method of claim 3, wherein the directed elicitation action comprises at least one of:
requesting the patient to produce a voluntary cough;
requesting the patient to speak a particular word or phrase selected to isolate respiratory acoustic artifacts;
requesting the patient to assume a particular physical position to redistribute fluid in airways;
requesting the patient to reposition a microphone-bearing device to improve audio capture quality;
requesting the patient to move to a new location having less ambient noise than a current location;
requesting the patient to switch to a higher-bandwidth communication medium to improve an audio quality of the artifact stream; or
any combination thereof.

5. The method of claim 1, further comprising:
receiving, by the second AI agent, contextual conversational information from the first AI agent, the contextual conversational information comprising at least one of a conversation transcript, a conversation summary, or an electronic health record data of the patient; and
including, by the second AI agent, the contextual conversational information in the analysis of the artifact stream to detect the respiratory acoustic event.

6. The method of claim 1, further comprising:
receiving biometric data from a wearable device associated with the patient; and
correlating, by the second AI agent, the biometric data with the respiratory acoustic event to produce an enhanced event prediction having improved diagnostic confidence.

7. The method of claim 1, wherein the respiratory acoustic event comprises one or more of: a spontaneous cough, a cough subtype, a wheeze, a stridor, a crackle, an involuntary breath hold, an abnormal breathing pattern, a voluntary cough quality assessment, or a positional artifact.

8. A server comprising:
one or more processors; and
one or more computer-readable storage media storing instructions executable by the one or more processors to perform operations comprising:
receiving an audio stream from a patient;
separating the audio stream into a speech stream and an artifact stream;
processing, by a first artificial intelligence (AI) agent, the speech stream to conduct a conversation with the patient;
performing an analysis of the artifact stream, by a second AI agent operating in parallel with the first AI agent, to detect a respiratory acoustic event;
determining, by the second AI agent, an event prediction comprising one or more of an event indicator, an event type classification, an event duration, a confidence score, or a severity score for the respiratory acoustic event;
applying, by a gating mechanism, one or more gating rules to the event prediction to determine an output action; and
based on determining that the output action satisfies an escalation rule, escalating the conversation by:
transferring the conversation to a medical professional;
automatically initiating a call to emergency services; or
both.

9. The server of claim 8, wherein separating the audio stream into the speech stream and the artifact stream comprises:
applying voice activity detection (VAD) to identify voiced speech segments in the audio stream;
routing voiced speech segments to the speech stream;
routing acoustic artifact segments to the artifact stream;
routing segments that include both voiced speech and acoustic artifacts to both the speech stream and the artifact stream.

10. The server of claim 8, wherein the second AI agent comprises a large language model (LLM) configured to:
analyze segments of the artifact stream and contextual conversational information; and
determine the event prediction comprising an event presence indicator, the event type classification, the event duration, the confidence score, and the severity score,
wherein the event type classification comprises at least one of: a spontaneous cough, a cough subtype, a wheeze, a stridor, a crackle, a rhonchus, an involuntary breath hold, an abnormal breathing pattern, or any combination thereof.

11. The server of claim 8, the operations further comprising:
monitoring audio quality metrics of the artifact stream, the audio quality metrics comprising at least one of a signal-to-noise ratio, a frequency bandwidth, or a compression artifact level;
determining that the audio quality metrics are below an audio quality threshold; and
instructing the patient to switch to a higher-bandwidth communication medium to improve an audio quality of the artifact stream.

12. The server of claim 8, wherein the escalation rule is based on at least one of:
the event type classification satisfying a subtype-specific escalation criterion;
a cumulative count of respiratory acoustic events within a time window exceeding an event count threshold;
the severity score exceeding a severity threshold; or
a combination of detected event subtypes satisfying a clinical pattern criterion.

13. The server of claim 8, the operations further comprising:
maintaining a respiratory event log comprising, for each detected respiratory acoustic event, the event type classification, the confidence score, the severity score, an event timestamp, and the event duration; and
outputting at least a portion of the respiratory event log in a form suitable for input to an electronic health records (EHR) platform.

14. The server of claim 8, the operations further comprising:
receiving biometric data from a wearable device worn by the patient; and
correlating, by the second AI agent, the biometric data with the respiratory acoustic event to generate an enhanced event prediction.

15. One or more non-transitory computer-readable storage media storing instructions executable by one or more processors to perform operations comprising:
receiving an audio stream from a patient;
separating the audio stream into a speech stream and an artifact stream;
processing, by a first artificial intelligence (AI) agent, the speech stream to conduct a conversation with the patient;
performing an analysis of the artifact stream, by a second AI agent operating in parallel with the first AI agent, to detect a respiratory acoustic event;
determining, by the second AI agent, an event prediction comprising one or more of an event indicator, an event type classification, an event duration, a confidence score, or a severity score for the respiratory acoustic event;
applying, by a gating mechanism, one or more gating rules to the event prediction to determine an output action; and
based on determining that the output action satisfies an escalation rule, escalating the conversation by:
transferring the conversation to a medical professional;
automatically initiating a call to emergency services; or
both.

16. The one or more non-transitory computer-readable storage media of claim 15, the operations further comprising:
applying a temporal suppression rule that prevents the gating mechanism from triggering a response to a respiratory event that occurs within a suppression window following a prior trigger; and
applying a cool down interval rule that enforces a minimum time interval between consecutive directed elicitation actions of a same type.

17. The one or more non-transitory computer-readable storage media of claim 15, the operations further comprising:
sending an elicitation instruction from the second AI agent to the first AI agent, the elicitation instruction causing the first AI agent to incorporate the elicitation instruction into the conversation to request the patient to perform a directed elicitation action; and
analyzing audio produced by the patient during the directed elicitation action to produce an updated event prediction.

18. The one or more non-transitory computer-readable storage media of claim 17, wherein the directed elicitation action comprises at least one of:
requesting the patient to produce a voluntary cough;
requesting the patient to speak a word or phrase selected to isolate respiratory acoustic artifacts in the artifact stream;
requesting the patient to assume a physical position to alter a distribution of fluid in the patient;
requesting the patient to reposition a device used by the patient to improve microphone proximity to a particular area of the patient;
requesting the patient to move to a new location having less ambient noise than a current location of the patient;
requesting the patient to switch to a higher-bandwidth communication medium; or any combination thereof.

19. The one or more non-transitory computer-readable storage media of claim 15, the operations further comprising:
maintaining a respiratory event log comprising, for each respiratory acoustic event, the event type classification, the confidence score, the severity score, an event timestamp, and the event duration, wherein the respiratory acoustic event comprises one or more of: a spontaneous cough, a cough subtype, a wheeze, a stridor, a crackle, an involuntary breath hold, an abnormal breathing pattern, a voluntary cough quality assessment, or a positional artifact.

20. The one or more non-transitory computer-readable storage media of claim 15, the operations further comprising:
receiving, by the second AI agent, contextual conversational information from the first AI agent, the contextual conversational information comprising at least one of a conversation transcript, a conversation summary, or an electronic health record data of the patient; and
including, by the second AI agent, the contextual conversational information in the analysis of the artifact stream to detect the respiratory acoustic event.
